(19) United States
(12) Patent Application Publication
(10) Pub. No.: US 2002/0151004 A1
(43) Pub. Date: Oct. 17, 2002

(54) Delivery vehicles and methods for using the same

(76) Inventor: Roger Craig, Sandbach (GB)

Correspondence Address: PALMER & DODGE, LLP, KATHLEEN M. WILLIAMS, 111 HUNTINGTON AVENUE, BOSTON, MA, 02199, US

(21) Appl. No.: 09/785,802

(22) Filed: Feb. 16, 2001

Related U.S. Application Data
(63) Continuation-in-part of application No. 09/748,789, filed on Dec. 22, 2000.
(63) Continuation-in-part of application No. 09/748,063, filed on Dec. 22, 2000.

(30) Foreign Application Priority Data
Aug. 9, 2000 (GB) 00/03056
Jul. 24, 2000 (GB) 00/02848

(51) Int. Cl. C12N 13/00; C12N 5/08

(52) U.S. Cl. 435/173.1; 424/93.7

(57) ABSTRACT

The invention provides delivery vehicles for the intracellular delivery of a therapeutic agent to a target site. The delivery vehicles comprise cells loaded with an agent conjugated to an MTS sequence. Selective release of the agent-MTS conjugate at a target site, facilitates the uptake of the agent by cells at the target site. Method for producing the cells and using the cells are also provided, as are kits to facilitate performing the methods.

### RELATED APPLICATIONS

This application claims priority under 35 U.S.C. §120 to U.S. patent application Ser. No. 09/748,063, and U.S. patent application Ser. No. 09/748,789, both filed Dec. 22, 2000. The entireties of these applications are incorporated by reference herein.

### FIELD OF THE INVENTION

The present invention relates to delivery vehicles for delivering an agent to a target tissue, and methods for using the same.

### BACKGROUND OF THE INVENTION

The delivery of therapeutic agents to specific tissues is desirable to ensure that a sufficiently high dose of a given agent is delivered to a selected biological target. In the case of nucleic acid- and protein-or peptide-based therapies, the biological target is typically intracellular and the therapeutic agent (e.g., an antibody, enzyme, transcription factor, peptide, nucleic acid and the like) is required not only to reach a selected tissue, but to traverse at least the cell membranes, and sometimes both the cell and nuclear membranes, of cells within the tissue. However, a limiting factor in the efficacy of nucleic acid- and protein/peptide-based therapies has generally been the low efficiency with which agents employed in these therapies cross cell membranes due to such factors as the intrinsic size of the agent, its charge, polarity and chemical composition.

A number of different methods have been developed for the delivery of agents into cells. For example, direct micro-injection of the agent into cells of interest may be used. Modified viruses have also been proposed as delivery vehicles or vectors. For example, viruses such as adeno associated virus (AAV), adenovirus, baculovirus, retroviruses, modified Semliki Forest Virus (SFV), lentiviruses (such as HIV) and herpesvirus (such as Herpes Simplex Virus, HSV) have been used to deliver agents intracellularly in methods of gene therapy.

It has also been suggested that agents may be delivered intracellularly as by fusing or conjugating the agents to proteins capable of crossing or translocating the plasma membrane and/or the nuclear membrane of a target cell. Known protein domains and sequences having translocational activity include sequences from the HIV-1-trans-activating protein (Tat), Drosophila Antennapedia homeodomain protein and the herpes simplex-1 virus VP22 protein.

Generally, delivery methods have relied on the systemic administration of a therapeutic agent or on the direct delivery of the agent to a target tissue (e.g., such as by injection). Each of these techniques has its disadvantages, including waste and lack of selectivity in the delivery process, which can lead to unwanted side effects. In the case of injection-based delivery methods, delivery of a therapeutic agent is limited to sites which are accessible; thus, surgical intervention may be needed to target internal sites.

### SUMMARY OF THE INVENTION

The present invention seeks to overcome the problems associated with prior art methods for delivering therapeutic agents intracellularly. The invention is based on the discovery that it is possible to utilise a membrane translocation sequence (“MTS”) conjugated to an agents of interest, to load a cellular delivery vehicle, such as a red blood cell. In one embodiment, the invention provides a method for delivering a therapeutic agent comprising exposing a cell to an agent conjugated to an MTS such that the agent-MTS conjugate automatically loads itself into the cell which becomes a delivery vehicle for the agent. The invention further provides delivery vehicles for therapeutic agents loaded with agent-MTS conjugates which effectively retain the agent-MTS conjugates until the conjugates are delivered to a target cell.

In one embodiment, the delivery vehicles which are loaded with the agent-MTS conjugates are sensitised, and preferably, electrosensitised, to render the delivery vehicles more susceptible to disruption by exposure to an energy source (e.g., such as ultrasound). Upon disruption of the delivery vehicle (e.g., by lysis), the agent-MTS conjugates are released in an active state and are taken up by target cells in proximity to the disrupted delivery vehicle. The invention thus provides a method which enables the local release and delivery of a therapeutic agent which can be taken up by one or more target cells at a target site.

According one embodiment, a method of preparing a cellular delivery vehicle suitable for delivering an agent to a target site in a vertebrate is provided. The method comprises the steps of: (a) providing a cell; and (b) loading the cell with an agent-MTS conjugate, thereby producing a cellular delivery vehicle. In a preferred embodiment, the cell is a red blood cell.

Preferably, the method further comprises the step of sensitising the cellular delivery vehicle, whether before or after the loading step, to render the cell more susceptible to disruption by exposure to a stimulus compared to a cell which has not been sensitised. In one embodiment, the stimulus comprises exposure to an energy source, such as ultrasound. In one embodiment, the cell is loaded prior to sensitisation. In another embodiment, the cell is loaded after sensitisation.

In one embodiment, sensitisation is performed by exposing the cell to an energy source, such as a source of electrical energy. In this embodiment, the cell may be sensitised by applying an electric field to the cell. Preferably, the electric field has a field strength of from about 0.1 kVolts/cm to about 10 kVolts/cm under in vitro conditions. More preferably, the cell is sensitised by application of an electric pulse for between 1 μs and 100 milliseconds. Most preferably, the cell is sensitised in such a way as to be capable of being disrupted by exposure to ultrasound, while fewer than 20%, and preferably fewer than 10% of non-sensitised cells are disrupted. In one embodiment, the ultrasound is selected from the group consisting of diagnostic ultrasound, therapeutic ultrasound and a combination of diagnostic and therapeutic ultrasound. The applied ultrasound energy source is preferably at a power level of from about 0.05 W/cm² to about 100 W/cm².

In a further embodiment, the cell is pre-sensitised prior to loading so that it is capable of being loaded with a larger amount of agent (e.g., a 2-fold greater amount of agent) than a cell which has not been pre-sensitised. Preferably, the pre-sensitisation step comprises exposing the cell to an electric field and/or ultrasound. In still a further embodiment, the cell is pre-sensitised to enhance its loading, and sensitised to enhance its ability to release the agent in the presence of a stimulus at a target site (e.g., such as a tissue comprising target cells).

The membrane translocation sequence may be any sequence which enables the agent to cross the plasma membrane of a cell. Preferably, the agent is a fusion protein, in which a therapeutic polypeptide is fused to a membrane translocation sequence.

In a preferred embodiment of the invention, the membrane translocation sequence comprises a sequence selected from the group consisting of: the sequence of an HIV-1-trans-activating protein (Tat), the sequence of Drosophila Antennapedia homeodomain protein (Antp-HD), the sequence of Herpes Simplex-1 virus VP22 protein (HSV-VP22), the sequence of a signal-sequence-based peptide, and the sequence of a Transportan and Amphiphilic model peptide. The membrane translocation sequence may further comprise homologues of the any of the foregoing, and fragments, variants and mutants thereof having membrane translocational activity.

In a highly preferred embodiment of the invention, the membrane translocation sequence comprises the amino acid sequence GRKKRRQRRRPPQC, RQIKIWFQNRRMKWKK or RQIKIWFQNRRMKWKKC.

In one embodiment, the agent is a biologically active molecule selected from the group consisting of: a protein, a polypeptide, a peptide, a nucleic acid, a virus-like particle, a nanoparticle, a steroid, a proteoglycan, a lipid, a carbohydrate, and analogs, derivatives, mixtures, fusions, combinations or conjugates thereof. In one embodiment, the agent is a nucleic acid, which is selected from the group consisting of an oligonucleotide, an aptamer, a ribozyme, an antisense molecule, a triple-helix forming molecule, a gene or gene fragment, a regulatory sequence, a cDNA, including analogs, derivatives, and combinations thereof. In another embodiment, the agent to be delivered is conjugated, or fused to, or mixed or combined with an imaging agent.

The invention further provides a delivery vehicle for use in any of the above-described methods. In a preferred embodiment, the delivery vehicle is a red blood and the target tissue is any tissue which can be made accessible to the red blood cell.

The invention also provides a kit comprising a cell for generating a delivery vehicle according to the invention (i.e., a cell for loading with an agent-MTS conjugate) and an agent and MTS for conjugating to the agent. In one embodiment however, the kit is provided with an agent which has already been conjugated to the MTS. In another embodiment, the cell is pre-sensitised to enhance its ability to be loaded with the agent-MTS conjugate. In still another embodiment, the cell is sensitised to enhance its ability to be disrupted by a stimuli at a target site. In a further embodiment, the kit comprises a cell which has been loaded with the agent-MTS conjugate (i.e., a delivery vehicle), which may or may not be sensitised. In still a further embodiment, the kit and delivery vehicles comprise more than one type of agent-MTS conjugate.

In another embodiment, the invention provides a pharmaceutical composition comprising a delivery vehicle and a physiologically compatible buffer. In one embodiment, the delivery vehicle comprises a red blood cell loaded with an agent-MTS conjugate. In another embodiment, the delivery vehicle is sensitised to facilitate the release of the agent-MTS conjugate with which it is loaded at a target site.

In another aspect of the invention, a method of loading a cell with an agent is provided, the method comprising the steps of: (a) providing a cell; and (b) exposing the cell to an agent-MTS conjugate. In a preferred embodiment, the cell is a red blood-cell. In a further embodiment, a method of producing a therapeutic agent is provided comprising providing an agent to be delivered to a cell and conjugating the agent to a membrane translocation sequence to produce an agent-MTS conjugate.

In a further embodiment, a method for delivering an agent to a target site in a vertebrate is provided, comprising the steps of: (a) providing a sensitised cell; (b) loading the cell with an agent-MTS conjugate; (c) introducing the cell into a vertebrate; and (d) causing the agent-MTS conjugate to be released from the sensitised cell. In a preferred embodiment, the cell is a red blood cell.

According to a yet further embodiment of the invention, a method is provided for the immunization of an animal with an antigen, the method comprising the steps of: (a) providing a cell; (b) loading the cell with an antigen; (c) introducing the cell into a vertebrate; and (d) causing the agent to be released from the cell. Preferably, the cell is a red blood cell. In one embodiment, the cell is sensitised, and more preferably, is electrosensitised, to render the cell more susceptible to disruption by exposure to a stimulus than an unsensitised cell. Preferably, the cell is disruptable by exposure to ultrasound. Preferably, steps (c) and/or (d) are repeated. In one embodiment, the antigen is conjugated to an MTS.

### BRIEF DESCRIPTION OF THE DRAWINGS

The invention will now be described by means of a description of various preferred non-limiting embodiments, with reference to the Figures, in which:

FIG. 1A is a diagram showing the loading of electrosensitised rabbit cells with an FITC-labelled HIV-TAT fragment. 1=control; 2=0 mg/ml; 3=0.05 mg/ml; 4=0.1 mg/ml; 5=0.2 mg/ml; 6=0.3 mg/ml; 7=0.4 mg/ml; 8=0.5 mg/ml.

FIG. 1B is a diagram showing the loading of electrosensitised rabbit cells with FITC-labelled penetratin. 1=0 mg/ml; 2=0.01 mg/ml; 3=0.03 mg/ml; 4 0.06 mg/ml; 5=0.1 mg/ml.

FIG. 1C is a diagram showing the loading of electrosensitised rabbit cells with FITC-labelled VP-22. 1=control; 2=0 mg/ml; 3=0.1 mg/ml; 4=0.2 mg/ml; 5=0.3 mg/ml; 6=0.4 mg/ml; 7=0.5 mg/ml.

FIG. 2A is a diagram showing the stability of HIV-TAT fragment loaded human cells in whole blood.

FIG. 2B is a diagram showing the stability of HIV-TAT fragment loaded rabbit cells in whole blood.

FIG. 2C is a diagram showing the stability of HIV-TAT fragment loaded pig cells in whole blood.

FIG. 2D is a diagram showing the stability of HIV-TAT fragment loaded mouse cells in whole blood.

FIG. 3 is a diagram showing an FL1 Dot Blot of Lymphocytes population: Lymphocytes (Red), Green (Penetratin 0.1 mg/ml and white Blood cells); Blue (ultrasound Lysate from RBC loaded Penetratin, Conc. 0.1 mg/ml).

FIG. 4 is a diagram showing the loading of FITC-labelled penetratin-oligonucleotide conjugate into sensitised human red blood cells.

FIG. 5 shows dialysis loading of an HIV-TAT fragment in pig erythrocytes. X-axis: FLH-1; Y axis: counts.

FIGS. 6A and 6B illustrate the stability of a loaded cell delivery vehicle according to one embodiment of the invention in whole blood. X-axis: time in hours; Y axes: percentage cells and geometric mean.

FIG. 6A: 4° C., 0.05 mg/ml 2^{nd} population;

FIG. 6B: 37° C., 0.05 mg/ml 2^{nd} population.

FIG. 7A shows events in the M2 region from electrosensitised, dialysed, HIV-TAT fragment-loaded pig cells subjected to varying ultrasound intensities in the circulating phantom. X-axis: time in minutes; Y-axis: events in the M2 region.

FIG. 7B shows haemoglobin release from electrosensitised, dialysed HIV-TAT fragment-loaded pig cells subjected to varying ultrasound intensities in the circulating phantom. X-axis: time in minutes; Y-axis: OD at 540 nm.

FIG. 7C shows haemoglobin release from non-electrosensitised, dialysed HIV-TAT fragment-loaded pig cells subjected to varying ultrasound intensities in the circulating phantom. X-axis: time in minutes; Y-axis: OD at 540 nm.

FIG. 8A is a graph showing ultrasound-mediated release of a peptide payload in vivo according to one embodiment of the invention. Arrows above denote 10 minute applications of ultrasound pulsed wave (35%) at 6 W/cm².

FIG. 8B illustrates the effect of ultrasound on electrosensitised loaded cells recovered from a pig 10 minutes post administration. X-axis: time in circulating phantom at 6 W/cm²; Y-axis: cells in M1 region.

FIG. 9 shows the effect of ultrasound on TAT-FITC loaded non-electrosensitised cells.

### DETAILED DESCRIPTION OF THE INVENTION

The invention provides delivery vehicles for the intracellular delivery of a therapeutic agent to a target site and methods for using the same. The delivery vehicles comprise cells loaded with an agent conjugated to an MTS sequence which enables the agent to cross the plasma membrane of a target cell. Selective release of the agent-MTS conjugate at a target site facilitates the uptake of the agent by cells at the target site.

### Definitions

In order to more clearly and concisely describe and point out the subject matter of the claimed invention, the following definitions are provided for specific terms which are used in the following written description and the appended claims.

As used herein, the term “loading” refers to introducing into a cell, such as a red blood cell, at least one agent. In a preferred embodiment, the agent is loaded by becoming internalised into the cell. Loading of a cell with more than one agent may be performed such that the agents are loaded individually (in sequence) or together (simultaneously or concurrently). Loading is generally performed in a separate procedure than sensitising. Agents may be first admixed at the time of contact with the cells or prior to that time. A “suitable period of time until the cell is loaded” refers to a time period after which there is no further increase in the amount of uptake of an agent.

The term “sensitised” is intended to indicate that the cells according to the invention have been treated in order to render them more susceptible to a stimulus. The term “sensitisation” as used herein, refers to the destabilisation of cells without causing fatal damage to the cells. As used herein, “destabilization” refers to an alteration of a membrane of a cell that makes the cell more susceptible to lysis in vitro or in vivo upon exposure to an energy field such as ultrasound. In one embodiment of the invention, a cell which is destabilized is a cell which is lysed when less than 20%, and preferably less than 5%-10%, or less than 1% of non-sensitised cells are lysed. Destabilisation may be achieved by exposing a cell, such as a red blood cell to an energy field, such as an electric field.

The term “electrosenitisation” as used herein refers to the sensitisation of a cell that occurs upon momentary exposure of the cell to one or more pulses of a high electric field. Electrosensitisation typically involves the use of electric fields which do not possess sufficient energy to electroporate cells. Electroporation, which facilitates the passage of agents into a cell without significant loss of cellular contents or cell viability is well known in the art, and apart from the energy levels involved is similar to electrosensitisation. Cells which are electroporated may become electrosensitised, However, as the term is used in the instant application, electrosensitisation is carried out at energy levels insufficient to electroporate a cell and permit the passage of substances through the cell membrane.

As used herein, the term “pre-sensitisation” refers to enhancing the efficiency of loading an agent into a cell, such as a red blood cell, compared to a cell which has not been subjected to pre-sensitisation. In one embodiment, loading efficiency is increased at least two-fold, 5-fold, 10-fold, 50-fold, or 100-fold compared to non-pre-sensitised cells. The term “pre-sensitisation” encompasses the destabilisation of cells without causing fatal damage to the cells. As used herein, a pre-sensitisation condition, is any condition to which a cell can be exposed which increases loading efficiency of the cell in comparison to a cell which is not pre-sensitised.

As used herein, the term “electric pulse” includes one or more pulses at variable capacitance and voltage and including exponential and/or square wave and/or modulated wave forms.

The term “resealing” encompasses the stabilization of the membrane of a cell by closing pores in the membrane that have previously been opened by some other process, for example, by a loading process such as hypotonic dialysis.

As used herein, the term “delivery vehicle” refers to a cell which has been loaded with an agent-MTS conjugate according to the invent.

As used herein, the term “red blood cell delivery vector” means a red blood cell that has been loaded, or is capable of being loaded, with one or more agent-MTS conjugate(s) according to the methods of the invention and can be used to deliver the agent to a vertebrate.

As used herein, the term “red blood cell” (RBC) refers to a living, enucleate red blood cell (i.e., a mature erythrocyte) of a vertebrate.

As used herein, the term “mammal” refers to a member of the class Mammalia including, but not limited to, a rodent, lagomorph, pig or primate. More preferably, the animal is selected from the group consisting of: mouse, rat, rabbit, sheep, goat, horse, cow, and pig. Most preferably, the mammal is a human.

The term “target site” refers to the site to which the delivery vehicle or cell loaded with a biological effector molecule will be delivered.

As used herein, the term “agent” includes, but is not limited to, an atom or molecule, inorganic or organic, which is a biological effector molecule or which encodes a biological effector molecule, and or which is a diagnostic molecule whose presence within a cell can be detected.

As used herein, the term “biological effector molecule” or “biologically active molecule” refers to an agent that has activity in a biological system.

As used herein, an “imaging agent” or a “diagnostic molecule” is an agent which may be detected, whether in vitro or in vivo in the context of a tissue, organ or organism in which the agent or molecule is located.

As used herein, the term “agent-MTS conjugate” refers to an agent which is coupled to a membrane translocation sequence or “MTS”. Coupling may be permanent or transient and may involve covalent or non-covalent interactions (including ionic interactions, hydrophobic forces, Van der Waals interactions, etc). The exact mode of coupling is not important, so long as the membrane translocation sequence is effective in allowing the agent to cross the cell membrane of a target cell. Accordingly, where reference is made to “comprising,” “conjugation,” “coupling,” “joining” etc, these references should be taken to include any form of interaction between the agent to be delivered and the membrane translocation sequence, in such a manner as to allow intracellular delivery of the agent. This term also includes fusion proteins comprising a membrane translocation sequence and a polypeptide agent to be delivered. In some embodiments, the MTS sequence may further comprises a nuclear localization sequence or a localization sequence which further directs the agent into a specific subcellular compartment.

As used herein, the term “nucleic acid” is defined to encompass DNA and RNA or both synthetic and natural origin which DNA or RNA may contain modified or unmodified deoxy- or dideoxy-nucleotides or ribonucleotides or analogues thereof. The nucleic acid may exist as single- or double-stranded DNA or RNA, an RNA/DNA heteroduplex or an RNA/DNA copolymer, wherein the term “copolymer” refers to a single nucleic acid strand that comprises both ribonucleotides and deoxyribonucleotides.

The term “synthetic,” as used herein, is defined as that which is produced by in vitro chemical or enzymatic synthesis.

As defined herein, a “modified nucleic acid,” or “modified oligonucleotide” refers to nucleic acids and oligonucleotides that contain non-naturally occurring nucleotides.

“Nanoparticles” are defined as solid colloidal particles ranging in size from about 10 nm to 1000 nm.

As used herein, the term “mixing” with reference to agents and/or MTS sequences refers to providing such agents and/or MTS sequences as separate molecules in a delivery vehicle.

As used herein, the term “combining” refers to providing a plurality of agents and/or MTS sequences as part of a single molecule.

As used here, the term ‘translocation’ refers to transfer of an agent across a membrane such that the agent is internalized within a cell.

As used herein, the term “fragment of an MTS sequence” or a “sub-sequence of an MTS sequence” refers to a polypeptide or peptide (or nucleic acid encoding the same) which comprises the biological activity of the MTS sequence, i.e., retains the ability to translocate an agent to which it is coupled across a cell and/or nuclear membrane or retains the ability to encode a polypeptide or peptide which can translocate an agent across a cell and/or nuclear membrane.

The term “variant of an MTS sequence” or a “mutated MTS sequence” refers to an MTS with one or more amino acid substitutions, deletions or insertions, or a nucleic acid sequence encoding an MTS with one or more substitutions, deletions or insertions which nevertheless retains MTS activity, i.e., the ability to translocate an agent to which it is coupled into a specific subcellular compartment.

The term “homolog of an MTS sequence” refers to a sequence which has at least 60% percent of its amino acid residues identical with the residues in a reference MTS sequence after aligning the two sequences and introducing gaps, if necessary, to achieve the maximum percent homology, using any computer program which is known in the art for performing the comparison, e.g., such as the GCG software package (available at http://www.gcg.com), the GAP program in the GCG software package (available at http://www.gcg.com), the NBLAST and XBLAST programs (version 2.0) of Altschul, et al. (1990) J. Mol. Biol. 215:403-10, or the Gapped BLAST program of Altschul et al., (1997) Nucleic Acids Res. 25(17):3389-340 (the entireties of these references are incorporated herein by reference). In a preferred embodiment, a homolog is at least 70% identical, at least 80% identical, or at least 90% identical, to a reference MTS sequence. A “homolog of an MTS sequence” as used herein has the ability to translocate an agent to which it is conjugated across a cell membrane.

As used herein the term “introducing” includes but is not limited to the administration of delivery vehicle and/or an agent into a vertebrate. As used herein in reference to administration of an agent to a vertebrate, the term “introducing” includes but is not limited to causing the agent to enter the circulatory system of the vertebrate by transfusion or to infusing an agent to a target site.

As used herein, the term “ultrasound” refers to a form of energy which consists of mechanical vibrations the frequencies of which are so high they are above the range of human hearing.

Agents

A variety of different agents may be conjugated with MTS's according to the invention.

In one embodiment of the invention, the agent is a biological effector molecule selected from the group consisting of a protein, polypeptide (a protein fragment greater in size than a peptide), a peptide (e.g., a 2-100 amino acid sequence), an antibiotic, a non-peptide (e.g., steroid) hormone; a proteoglycan; a lipid; a carbohydrate, a nucleic acid, a purine analogue, a pyrimidine analogue, a chemotherapeutic agents a virus or virus-like particle, and a nanoparticle. These agents frequently present drug delivery problems. Small molecules, including organic and inorganic chemicals are also of use in the present invention. In a particularly preferred embodiment of the invention, the biologically active agent is a pharmaceutically active agent, for example, an isotope.

In one embodiment, the biological effector molecule comprises a nucleic acid selected from the group consisting of an oligonucleotide or modified oligonucleotide, an antisense oligonucleotide or modified antisense oligonucleotide, an aptamer, a cDNA, genomic DNA (including gene sequences or fragments thereof and/or regulatory sequences), an artificial or natural chromosome (e.g., a yeast artificial chromosome) or a part thereof, defibrinotide molecules, RNA, including an mRNA, tRNA, rRNA or a ribozyme (e.g., such as a hammerhead ribozyme, as disclosed in Sullivan, 1994, J. Invest. Dermatol., 103: 85S-98S; Usman et al, 1996, Curr. Opin. Struct. Biol., 6: 527-533, the entireties of which are incorporated by reference herein), or a peptide nucleic acid (PNA), and/or a vector comprising any of the preceding (e.g., such a viral or non-viral DNA or RNA vector, where non-viral vectors include, but are not limited to, plasmids, linear nucleic acid molecules, artificial chromosomes, condensed particles, episomal vectors, and the like).

While in one embodiment, the nucleic acid itself is a biological effector molecule, in another embodiment, the nucleic acid encodes a biological effector molecule. In this embodiment, the nucleic acid sequence encoding the agent can be operatively linked to transcriptional and translational regulatory elements active in a cell at the target site, suitable for driving the expression of a heterologous gene (see, e.g., as described in Wolff J. A. et al., 1990, Science, 247: 1465-1468; Carson D. A. et al., U.S. Pat. No. 5,580,859; Sykes et al., 1994, Human Gene Ther., 5: 837-844; Vile et al., 1993, Cancer Res., 53: 962-967; Hengge, et al, 1995, Nature Genet., 10: 161-166; Hickman, et al., 1994, Human Gene Therapy, 5: 1477-1483; and Meyer et al., 1995, Gene Therapy, 2: 450-460, the entireties of which are incorporated by reference herein).

Therapeutic nucleic acid sequences useful according to the methods of the invention include those encoding receptors, enzymes, ligands, regulatory factors, and structural proteins. Therapeutic nucleic acid sequences also include sequences encoding nuclear proteins, cytoplasmic proteins, mitochondrial proteins, secreted proteins, plasmalemma-associated proteins, serum proteins, viral antigens, bacterial antigens, protozoal antigens and parasitic antigens. Therapeutic nucleic acid sequences useful according to the invention also include sequences encoding proteins, lipoproteins, glycoproteins, phosphoproteins and nucleic acids (e.g., RNAs such as ribozymes or antisense nucleic acids).

Proteins or polypeptides which can be expressed by nucleic acid molecules delivered according to the present invention include neurotransmitters, enzymes, immunoglobulins, antibodies, toxins, apolipoproteins, receptors, drugs, oncogenes, tumour antigens, tumour suppressers, structural proteins, viral antigens, parasitic antigens and bacterial antigens. The compounds which can be incorporated are only limited by the availability of the nucleic acid sequence encoding a given protein or polypeptide. One skilled in the art will readily recognise that as more proteins and polypeptides become identified, their corresponding genes can be cloned into the gene expression vector(s) of choice, administered to a tissue of a recipient patient or other vertebrate, and expressed in that tissue.

In some embodiments, the agent is a nucleic acid base, or analogue or derivative thereof.

In one embodiment, the agent is one or more of adenine, guanine, cytosine and thymine are well, or analogue or derivative thereof, such as 6-mercaptopurine (6 MP) and azathioprine, which are commonly used as immunosuppressants and inhibitors of malignant cell growth, and azidothymidine (AZT) and analogues thereof which are useful as anti-viral agents, particularly in the treatment of AIDS. It has been shown that the action of these unnatural base derivatives is dependent on intra-cellular conversion thereof to phosphorylated forms (Chan et al., 1987, Pharmacotherapy, 7: 165;14-77; also Mitsuya et al., 1986, Proc. Natl. Acad. Sci. U.S.A., 83: 1911-1915).

In another embodiment, the biological effector molecule comprises a protein or fragment thereof having biological activity. In one embodiment, the protein is selected from the group consisting of a structural protein, an enzyme, a cytokine (such as an interferon and/or an interleukin), a polyclonal or monoclonal antibody, or an effective part thereof, such as an Fv fragment, which antibody or part thereof, may be natural, synthetic or humanised, a peptide hormone, a receptor, or a signalling molecule. Included within the term “immunoglobulin” are intact immunoglobulins as well as antibody fragments such as Fv, a single chain Fv (scFv), a Fab or a F(ab′)₂. Preferred immunoglobulins, antibodies, Fv fragments, etc, are those which are capable of binding to antigens in an intracellular environment, known as “intrabodies” or “intracellular antibodies.” An “intracellular antibody” or an “intrabody” is an antibody which is capable of binding to its target or cognate antigen within the environment of a cell, or in an environment which mimics an environment within the cell.

Selection methods for directly identifying such “intrabodies” include the use of an in vivo two-hybrid system for selecting antibodies with the ability to bind to antigens inside mammalian cells. Such methods are described in International Patent Application number PCT/GB00/00876, incorporated herein by reference. Techniques for producing intracellular antibodies, such as anti-β-galactosidase scFvs, have also been described in Martineau et al., 1998, J Mol Biol., 280, 117-127 and Visintin et al., 1999, Proc. Natl. Acad. Sci. USA 96, 11723-11728, the entireties of which are incorporated herein.

In another embodiment, the biological effector molecule is an antigen which is used to stimulate an immune response. In a further embodiment, the immune response is a protective immune response such as a vaccine response.

In yet another embodiment, the agent is an amino acid compound such as tryptophan, phenylalanine, other water-soluble amino acid compounds, and the like.

In a further embodiment, combinations of biological effector molecules are provided. For example, in one embodiment, a combination of nucleic acid and protein is provided (e.g., such as chromosomal material comprising both protein and DNA components), or a pair or a set of effector molecules, wherein one or more of the molecules within the set convert one or more molecules within the set from an inactive to an active form (e.g., catalytically).

Other particularly useful classes of biological effector molecules include, but are not limited to, antibiotics, anti-inflammatory drugs, angiogenic or vasoactive agents, growth factors and cytotoxic agents (e.g., tumour suppressers). Cytotoxic agents of use in the invention include, but are not limited to, diptheria toxin, Pseudomonas exotoxin, cholera toxin, pertussis toxin, and the prodrugs peptidyl-p-phenylenediamine-mustard, benzoic acid mustard glutamates, ganciclovir, 6-methoxypurine arabinonucleoside (araM), 5-fluorocytosine, glucose, hypoxanthine, methotrexate-alanine, N-[4-(a-D-galactopyranosyl) benyloxycarbonyl]daunorubicin, amygdalin, azobenzene mustards, glutamyl p-phenylenediamine mustard, phenolmustard-glucuronide, epirubicinglucuronide, vinca-cephalosporin, phenylenediamine mustard-cephalosporin, nitrogen-mustard-cephalosporin, phenolmustard phosphate, doxorubicin phosphate, mitomycin phosphate, etoposide phosphate, palytoxin-4-hydroxyphenyl-acetamide, doxorubicinphenoxyacetamide, melphalan-phenoxyacetamide, cyclophosphamide, ifosfamide or analogues thereof.

In some embodiments, cells according to the invention are loaded with a prodrug. If a prodrug is loaded in inactive form, a second biological effector molecule may be loaded into the delivery vehicle of the present invention. Such a second biological effector molecule is usefully an activating polypeptide which converts the inactive prodrug to active drug form. Activating polypeptides encompassed within the scope of the present invention, include, but are not limited to, viral thymidine kinase (encoded by Genbank Accession No. J02224), carboxypeptidase A (encoded by Genbank Accession No. M27717), a-galactosidase (encoded by Genbank Accession No. M13571), β-glucuronidase (encoded by Genbank Accession No. M1 5182), alkaline phosphatase (encoded by Genbank Accession No. J03252 J035 12), or cytochrome P-450 (encoded by Genbank Accession No. D00003 N00003), plasmin, carboxypeptidase G2, cytosine deaminase, glucose oxidase, xanthine oxidase, β-glucosidase, azoreductase, t-glutamyl transferase, β-lactamase, or penicillin amidase.

Either the polypeptide or the gene encoding it may be loaded; if the latter, both the prodrug and the activating polypeptide may be encoded by genes on the same recombinant nucleic acid construct. Furthermore, either the prodrug or the activator of the prodrug may be transgenically expressed and already loaded into the red blood cell according to the invention. The relevant activator or prodrug (as the case may be) is then loaded as a second agent according to the methods described here.

In another embodiment, the agent is a diagnostic molecule. For example, in one embodiment, an agent is provided which is useful for imaging tissues in vivo or ex vivo. In this embodiment, the imaging agent can emit a detectable signal, such as light or other electromagnetic radiation. In another embodiment, the imaging agent is a radio-isotope, for example ³²P or ³⁵S or ⁹⁹Tc, or a molecule such as a nucleic acid, polypeptide, or other molecule, conjugated with such a radio-isotope. In one embodiment, the imaging agent is opaque to radiation, such as X-ray radiation. In another embodiment, the imaging agent comprises a targeting functionality by which it is directed to a particular cell, tissue, organ or other compartment within the body of an animal. For example, the agent may comprise a radiolabelled antibody which specifically binds to defined molecule(s), tissue(s) or cell(s) in an organism. In one embodiment, the imaging agent may be combined with, conjugated to, mixed with, or combined with, any of the other agents disclosed herein.

Membrane Translocation Sequences (MTS)

The present invention encompasses the use of polypeptide sequences or domains which are able to direct proteins, polypeptides, and other molecules across the cell membrane and into the cell. By coupling such sequences to therapeutic agents, conjugates are created which can be loaded into the delivery vehicles of the invention, to selectively target intracellular sites in need of the biological activity of these agents. The use of fragments or variants of sequences which comprise membrane translocational activity is also included, as are sub-sequences, variants, fragments, etc., of polypeptides capable of directing localization into subcellular compartments (such as the nucleus).

The presence of such sequences facilitates the intake of agent into a cell, and thus enables efficient intracellular delivery of agent. As explained above, one or more of these sequences may be coupled, fused, conjugated, or otherwise joined, to the agent to be delivered in order to effect intracellular delivery of the agent-MTS conjugate. In a highly preferred embodiment of the invention, polypeptides for delivery are expressed as fusion proteins with one or more membrane translocation sequences.

There appears to be no restriction on the type of molecule that can penetrate cell membranes when fused to protein translocation sequences. Therefore, the method of our invention may be used for the in vivo intracellular delivery of a wide variety of agents. For example, Fawell et al. 1994, Proc. Natl. Acad. Sci. USA., 91: 664-668 demonstrate that fusion proteins comprising MTS's can enter tissues in vivo in mice. Pooga et al., 1998, Nat. Biotechnol., 16: 857-861 demonstrate that fusions can penetrate the blood-brain barrier in rats. Many different protein translocation sequences have now been identified that can penetrate the cell membrane (reviewed by Lindgren et al. 2000, Trends Pharma. Sci., 21: 99-103; Morris, et al., 2000, Curr. Opin. Biotech. 11: 461-466; Hawiger, 1999, Curr. Opin. Chem. Biol., 3: 89-94).

Preferred membrane translocation sequences include the whole sequence or subsequences of the HIV-1-trans-activating protein (Tat), Drosophila Antennapedia homeodomain protein (AntpHD), Herpes Simplex-1 virus VP22 protein (HSV-VP22), signal-sequence-based peptides, Transportan and Amphiphilic model peptide, among others. These membrane translocation sequences, as well as domains and sequences from them which are useful for in the present invention, are described in further detail below.

HIV-1-Trans-Activating Protein (Tat)

The Human Immunodeficiency Virus trans-activating protein (Tat) is a 86-102 amino acid long protein involved in HIV replication. Exogenously added Tat protein can translocate through the plasma membrane to reach the nucleus, where it transactivates the viral genome. Intraperitoneal injection of a fusion protein consisting of β-galactosidase and Tat results in delivery of the biologically active fusion protein to all tissues in mice (Schwarze et al., 1999, Science, 285:1569-72). Methods of delivering molecules such as proteins and nucleic acids into the nucleus of cells using Tat or Tat-derived polypeptides are described in detail in U.S. Pat. No.

Nos. 5,652,122, 5,670,617, 5,674,980, 5,747,641 and 5,804,604, the entireties of which are incorporated herein by reference.

Vives et a., 1997, J. Biol. Chem., 272: 16010-7, identified a sequence of amino acids 48-60 (CGRKKRRQRRRPPQC) from Tat important for translocation, nuclear localization and trans-activation of cellular genes. This core sequence also includes a nuclear localization sequence and has been found to exhibit translocational activity. Accordingly, our invention encompasses the use of polypeptides comprising the entire HIV-Tat sequence as well as polypeptides comprising the core sequence for translocating an agent into a cell. It will however be appreciated that variations about the core sequence, such as shorter or longer fragments (such as for example amino acids 47-58), may also possess translocational activity, and that these sequences may also be usefully employed.

To date, numerous Tat derived short membrane translocation domains and sequences have been identified that possess transtocation activity; furthermore, translocation has been found to occur in various different cell types (Lindgren et al., 2000, Trends Pharma. Sci., 21: 99-103). Examples of fragments which possess translocational activity include amino acids 37-72 (Fawell et al., 1994, Proc. Natl. Acad. Sci. USA., 91: 664-668), 37-62 (Anderson et al., 1993, Biochem. Biophys. Res. Commun., 194: 876-884) and 49-58 (having the basic sequence RKKRRQRRR). Any of these fragments may be used alone or in combination with each other, and/or preferably with the core sequence, to enable translocation of an agent into a cell.

Internalization of Tat is though to occur by endocytosis (Frankel and Pabo, 1988, Cell, 55: 1189-1193). Co-administration of basic peptides such as protamine or Tat fragments (amino acids 38-58) has been found to stimulate Tat uptake into cells. Accordingly, the present invention also encompasses the use of these, and other agents, which stimulate uptake (“translocation enhancers”) to enhance the delivery of an agent into a cell. Use of such translocation enhancers need not necessarily be restricted to enhancing translocation of Tat conjugates/fusions—our invention encompasses the use of such enhancers to enhance delivery of conjugates and/or fusions with other membrane translocation sequences (and/or fragments or domains of these), as described below. Thus, one or more translocation enhancers may be administered to the recipient before, after or at the same time as the loaded red blood cells are administered. Alternatively, the red blood cell may be loaded with the translocation enhancer(s) as well as the agent preferably joined to a membrane translocation sequence, to be delivered. Disruption of the red blood cell at the point of delivery releases both the agent to be delivered and the translocation enhancer, thus stimulating uptake of the agent by the target cell or tissue, etc.

Tat-derived polypeptides lacking the cysteine rich region (22-36) and the carboxyl terminal domain (73-86) have been found to be particularly effective in translocation. Absence of the cysteine rich region and the carboxy terminal domain prevents spurious trans-activation and disulfide aggregation. In addition, the reduced size of the transport polypeptide minimizes interference with the biological activity of the molecule being transported and increases uptake efficiency. Such polypeptides are used in the methods described in U.S. Pat. Nos. 5,652,122, 5,670,617, 5,674,980, 5,747,641 and 5,804,604, the entireties of which are incorporated by reference herein. Accordingly, the present invention encompasses the use of such Tat-derived polypeptides lacking the carboxyl terminal domain and/or the cysteine rich-region to improve the efficiency of translocation. Preferably, the Tat-derived polypeptide lacks amino acids 73-86 of the Tat protein or amino acids 73-86 of the Tat protein. More preferably, the membrane translocation sequence comprises a Tat-derived protein which lacks both domains.

Drosophila Antennapedia Homeodomain Protein (Antp-HD)

Agents may be conjugated or fused with all or part of the Drosophila Antennapedia homeodomain protein, preferably, the third helix of Antp-HD, which also has cell penetration properties (reviewed in Prochiantz, 1999, Ann. N. Y. Acad. Sci., 886: 172-9). Cell internalization of the third helix of Antp-HD appears to be receptor- and endocytosis-independent. Derossi et al., 1996, J. Biol. Chem., 271: 18188-93, suggest that the translocation process involves direct interactions with membrane phospholipids.

The region responsible for translocation in Antp-HD has been localized to amino acids 43-58 (third helix), a 16 amino acid long peptide rich in basic amino acids having the sequence RQIKIWFQNRRMKWKK (Derossi et al., 1994, J. Biol. Chem., 269: 10444-50). This peptide is known as Penetratin® and has been used to direct biologically active substances to the cytoplasm and nucleus of cells in culture (Theodore et al., 1995, J. Neurosci. 15: 7158-7167). Chimeric peptides less than 100 amino acids and oligonucleotides up to 55 nucleotides are capable of being internalized. Thoren et al., 2000 FEBS Lett. 6: 265-8 show that Penetrating traverses a lipid bilayer, further supporting the idea that cell internalization of the third helix of Antp-HD is receptor- and endocytosis-independent. Our invention therefore encompasses the use of AntpHD or fragments of Antp-HD (including preferably fragments comprising, more preferably consisting of, RQIKIWFQNRRMKWKK, i.e., Penetratin) for intracellular delivery of agents.

Antp-HD and its fragments may be conjugated with proteins and nucleic acids by methods known in the art, for example as described in WO 99/11809, the entirety of which is incorporated by reference herein. WO 99/11809 also describes sequences homologous to AntpHD isolated from other organisms, including vertebrates, mammals and humans; homologs of Penetratin® are also described in EP 485578, the entirety of which is incorporated by reference herein. The present invention encompasses the use of these and other homologs and fragments of these for delivery of agents into cells. Truncated and modified forms of Antp-HD and Penetratin are described in WO 97/12912, UK 9825000.4 and UK 9902522.3, the entireties of which are incorporated by reference herein. For example, truncated polypeptides of 15 and 7 amino acids such as RRMKWKK have been found to be active in translocation. Accordingly our invention encompasses the use of such truncated and modified forms of Antp-HD and its homologs.

To improve intracellular delivery, Antp-HD and/or its fragments may be conjugated to peptide nucleic acid (PNA), as described by Nielsen et al., 1991, Science, 254: 1497-1500. PNA is resistant to proteases and nucleases and is much more stable in cells than regular DNA. Pooga et al., 1998, Nat. Biotechnol., 16: 857-861 show that a 21-mer PNA complementary to human galanin receptor mRNA, coupled to Antp-HD, is efficiently taken up into Bowes melanoma cells, thus suppressing the expression of galanin receptors. The invention therefore includes the use of conjugates and/or fusions of agents, membrane translocation proteins (and/or fragments) and peptide nucleic acid.

Herpes Simplex-1 Virus VP22 Protein

The VP22 tegument protein of herpes simplex virus also exhibits membrane translocation activity. Thus, VP22 protein expressed in a subpopulation of cells spreads to other cells in the population (Elliot and O'Hare, 1997, Cell, 88: 223-33). Fusion proteins consisting of GFP (Elliott and O'Hare, 1999, Gene Ther., 6: 149-51), thymidine kinase protein (Dilber, et al., 1999, Gene Ther 6:12-21) or p53 (Phelan et al., 1998, Nat Biotechnol. 16: 440-3) with VP22 have been targeted to cells in this manner.

HSV-VP22 has the amino acid sequence NAATATRGRSAASRPTERPRAPARSASRPRRPVE and agents may be conjugated or fused to this polypeptide (or fragments exhibiting translocation activity) for delivery into cells. As noted above, an important property of HSV-VP22 is that when applied to the surrounding medium, VP-22 is taken up by cells and accumulates in the nucleus. Thus, fusion proteins of HSV-VP22 conjugated to GFP (Elliott and O'Hare, 1999, Gene Ther., 6: 149-51), thymidine kinase protein (Dilber, et al., 1999, Gene Ther., 6: 12-21) and p53 (Phelan et al., 1998, Nat. Biotechnol., 16: 440-3) have been targeted to cells in this manner. The mechanism of transport is thought to be via a Golgi-independent pathway. Fusion proteins comprising HSV-VP22 (and sub-sequences) and a protein of interest, and the transport of such fusions into a cell are described in U.S. Pat. No. 6,017,735, the entirety of which is incorporated by reference herein.

Proteins capable of being transported by the methods described in U.S. Pat. No. 6,017,735 include those involved in apoptosis, suicide proteins and therapeutic proteins. A feature of HSV-VP22 is that it binds to microtubules in cells as described in WO 98/42742. Therefore, fusions, conjugates, etc of HSV-VP22 (including its fragments) with agents may be delivered into cells to stabilize microtubules and retard or enhance cell growth. Variants of VP22 may be prepared in which the potency of this property is altered. Agents which enhance or inhibit microtubule polymerization or depolymerization may be delivered to enhance or retard cell growth. Furthermore, HSV-VP22 fusions/conjugates may be employed where microtubule transport of an agent to a particular intracellular compartment or location is desired.

Signal-Sequence-Based Peptides

Signal sequences of peptides are recognized by acceptor proteins that aid in addressing the pre-protein from the translation machinery to the membrane of appropriate intracellular organelles. The core hydrophobic region of a signal peptide sequence may be used as a carrier for cellular import of relevant segments or motifs of intracellular proteins (Linet al, 1995, J Biol. Chem. 270: 14255-14258; Liu, et al., 1996, Proc Natl. Acad. Sci USA, 93: 11819-11824). Synthetic membrane translocation domains and sequences containing such hydrophobic regions are able to translocate into cells.

The hydrophobic region, also known as the h region, consists of 7-16 non-conserved amino acids, and has been identified in 126 signal peptides ranging in length from 18-21 amino acids (Prabhakaran, 1990, Biochem J., 269: 691-696). Any of these sequences may be employed in the present invention. Signal sequence based translocators are thought to function by acting as a leader sequence (“leading edge”) to carry peptides and proteins into cells (reviewed by Hawiger, 1999, Curr. Opin. Cell. Biol., 3: 89-94). Use of signal peptides for delivery of biologically active molecules is disclosed in U.S. Pat. No. 5,807,746, the entirety of which is incorporated by reference herein.

It is known that import of polypeptides comprising the signal sequence h-region does not require membrane caveolae (Torgerson et al., J. Immunol., 161: 6084-6092) or endosomal uptake (Lin et al., 1995, J. Biol. Chem., 270: 14255-14258; Hawiger, 1997, Curr. Opin. Immunol., 9:189-194) but requires an intact plasma membrane (Lin et al., 1995, J. Biol. Chem. 270: 14255-14258). Furthermore, the uptake mechanism is concentration- and temperature-dependent, independent of cell type and receptor. Signal sequence based peptides can translocate into a number of cell types that include five human cell types (monocytic, endothelial, T lymphocyte, fibroblast and erythroleukemia) and three murine lines. Accordingly, the invention encompasses the use of membrane translocation sequences, including signal sequence h-regions, conjugates, fusions, etc, for intracellular delivery of agents.

Membrane translocation sequences comprising signal sequence based peptides coupled to nuclear localization sequences (NLSs) may also be utilized. Thus, for example, the MPS peptide (Signal-sequence-based peptide I) is a chimera of the hydrophobic terminal domain of the viral gp41 protein and the NLS from the 5V40 large antigen (GALFLGWLGAAGSTMGAWSQPKKKRKV) (Morris et al., 1997, Nucleic Acids Res. 25: 2730-2736), and has been found to be active in membrane translocation. The peptide AAVALLPAVLLALLAP (Signal-sequence-based peptide II) is derived from the nuclear localization signal of NF-κB p50 (Lin et al., 1996, Proc. Natl. Acad. Sci. USA 93: 11819-11824) and USF2 (Frenkel et al., 1998, J. Immunol., 161, 2881-2887). A peptide having the sequence AAVLLPVLLAAP is derived from the Grb2 SH2 domain (Rojas et al., 1998, Nat. Biotechnol., 16: 370-375) and VTVLALGALAGVGVG from the Integrin β₃ cytoplasmic domain (Liu et al, 1996, Proc. Natl. Acad. Sci. USA, 93: 11819-11824). Peptides comprising membrane translocation sequence-nuclear localization sequence have been shown to enter several cell types.

Membrane translocation sequences derived from the hydrophobic regions of the signal sequences from Kaposi's sarcoma fibroblast growth factor 1 (K-FGF) Lin et al., 1995, J. Biol. Chem. 271: 5305-5308) and human β integrin (Liu et al., 1996, Proc. Natl. Acad. Sci. USA, 93: 11819-11824), the fusion sequence of HIV-1 gp41 (Morris et al., 1997, Nucleic Acid Res, 25: 2730-2736) and the signal sequence of the variable immunoglobulin light chain Ig(v) from Caiman crocodylus (Chaloin et al., 1997, Biochemistry, 36: 11179-11187) conjugated to NLS peptides originating from nuclear transcription factor κB (NF-κB) (Zhang et al., 1998, Proc Natl. Acad. Sci USA, 95: 9184-9189), SV40 T-antigen (Chaloin et al., 1998, Biochem. Biophys. Res. Commun., 243: 601-608) or K-FGF (Lin et al., 1995, J. Biol. Chem., 270: 14255-14258) may also be employed. Any of the peptides described above may be used alone or in combination, preferably in conjunction with nuclear localization sequences, to deliver fused or conjugated agents into a cell.

Transportan

Agents for delivery may be conjugated or fused or joined with transportan. Transportan comprises a fusion between the neuropeptide galanin and the wasp venom peptide mastoparan. It is found to be localized in both the cytoplasm and nucleus (Pooga, et al., 1998, FASEB J., 12: 67-77). Transportan comprises the sequence GWTLNSAGYLLKINLKALAALAKKIL. Transportan may be used as a carrier vector for hydrophilic macromolecules. Cell-penetrating ability is not restricted by cell type and seems to be a general feature of this membrane translocation domain. Cellular uptake is not inhibited by unlabeled transportan or galanin and shows no toxicity at concentrations of 20 μM or less. However, concentrations of 50 μM decrease GTPase activity (Pooga, et al., 1998, Ann. New York Acad Sci., 863: 45-453). The mechanism of cell penetration by transportan is not clear; however, it is known to be energy independent and that receptors and endocytosis are not involved. Deletion analogs of transportan have been prepared (Soomets, et al., 2000, Biochim. Biophys. Acta., 1467:165-176) to identify those regions of the sequence responsible for translocation. Deletion of six amino acids from the N-terminus of transportan does not impair cell penetration. Deletions at the C-terminus or in the middle of the protein decrease or abolish translocation activity. Accordingly, the invention includes the use of transportan, as well as deletions of transportan comprising translocation activity (preferably N-terminal deletions of 1, 2, 3, 4, 5 or 6 amino acids) in the delivery of agents into cells. The invention furthermore includes the use of novel short analogs disclosed by Lindgren, et al., 2000, Bioconjug Chem 11(5): 619-26, with similar translocation properties but with reduced undesired effects such as inhibition of GTPase activity.

Amphiphilic Model Peptide

Agents may be conjugated with amphiphilic model peptide. Amphiphilic model peptide is a synthetic 18-mer (KLALKLALKALKAALKLA) first synthesised by Oehlke, et al., 1998, Biochim. Biophys. Acta., 1414: 127-139. Analogues that show less toxicity and higher uptake have been synthesized by Scheller, et al, 1999, J. Peptide Sci., 5: 185-194. The only essential structural requirement for amphiphilic model peptides is a length of four complete helical turns. The membrane translocation sequence crosses the plasma membranes of mast cells and endothelial cells by both energy-dependent and -independent mechanisms. The uptake behavior shows analogy to several membrane translocation domain sequences including Antp-HD and Tat.

While it is clear from the above that any of the membrane translocation sequences (including domains and/or sequences and/or fragments of these exhibiting membrane translocation activity) may be used for the purpose of delivery of an agent into a cell, it should also be appreciated that other variations are also possible. For example, variations such as mutations, (including point mutations, deletions, insertions, etc) of any of the sequences disclosed here may be employed, provided that some membrane translocation activity is retained. Furthermore, it will be clear that any homologs of the membrane translocation proteins identified above, for example, from other organisms (as well as variations), may also be used.

Particular domains or sequences from proteins capable of translocation through the nuclear and/or plasma membranes may be identified by mutagenesis or deletion studies. Alternatively, synthetic or expressed peptides having candidate sequences may be linked to reporters and translocation assayed. For example, synthetic peptides may be conjugated to fluoroscein and translocation monitored by fluorescence microscopy by methods described in Vives, et al., 1997, J Biol. Chem., 272: 160 10-7. Alternatively, green fluorescent protein may be used as a reporter (Phelan et al., 1998, Na. Biotechnol., 16: 440-3).

The membrane translocation sequence may be linked to the agent to be delivered such that more than one agent can be delivered into a cell. The protein or fragment may contain components that facilitate the binding of multiple agents, for example drugs, such as naturally occurring or synthetic amino acids. In this manner up to 32 different agents can be linked to the membrane translocation sequence and delivered. Such a method of using a membrane translocation sequence to facilitate the transfer of drugs is described in detail in WO 00/01417, the entirety of which is incorporated by reference herein.

Generating Agent-MTS Conjugates

Agents may be fused to membrane translocation sequences, including nucleic acids, proteins, or fragments thereof, using a variety of methods. For example, using peptide synthesis, the membrane translocation sequence can be chemically synthesized and linked with any peptide sequence or chemical compound (Lewin, et al., 2000, Nat. Biotechnol., 18: 410-414) using methods well known in the art. Peptides can also be chemically cross-linked to larger peptides and proteins (Fawell, et al., 1994, Proc. Natl. Acad Sci. USA, 91: 664-668). Furthermore, fusion proteins comprising the polypeptide agent fused to a membrane translocation sequence may be expressed in any suitable host, for example, a bacterial host (Nagahara et al., 1998, Nat. Med., 4: 1449-1452). A cDNA encoding an agent of interest of interest may be constructed to include sequences encoding a membrane translocation protein or fragment as well, in-frame downstream of an N-terminal leader sequence, for example, a sequence comprising a 6-Histidine tag. This enables purification of the expressed recombinant fusion proteins using methods known in the art.

The agent(s) may also be chemically coupled, either directly or indirectly, to the membrane translocation proteins, fragments, etc. The coupling may be permanent or transient, and may involve covalent or non-covalent interactions. Coupling technologies are well known in the art.

Direct linkage may be achieved by means of a functional group on the agent such as a hydroxyl, carboxy or amino group. Indirect linkage can occur through a linking moiety such as, but not limited to, one or more of bi-functional cross-linking agents, as known in the art. In this manner, a second agent comprising such fusion and/or conjugate, etc to be easily loaded into a transgenic cell (i.e., a cell carrying a transgene), such as a red blood cell.

In a highly preferred embodiment of the invention, the agent-MTS conjugate is one which does not elicit an immune response, or one which elicits a minimal immune response, when the agent-MTS conjugate is exposed to the donor animal. Preferably, the membrane translocation sequence does not elicit, or elicits a minimal, immune response. Thus, preferably, the membrane translocation sequence may be derived from a mammalian source, or is otherwise a mammalian homologue of a membrane translocation sequence as disclosed above. Preferably, therefore, in relation to a human recipient, the membrane translocation sequence comprises a human transportan, a human amphiphilic model peptide, or a human signal-sequence-based peptide. In other words, a signal sequence from any known human protein may be used as the basis for designing a suitable translocation sequence.

In the alternative, the membrane translocation sequence may be a humanized membrane translocation sequence, the term being understood to mean a sequence in which one or more residues of a membrane translocation sequence are substituted with other residues to minimize an immune response when the agent-MTS conjugate is exposed to a human.

Polymer Conjugates

The agents may further be delivered attached to polymers, so long as either or both the agent and the polymer comprises a membrane translocation sequence. Polymer-based therapeutics have been proposed to be effective delivery systems, and generally comprise one or more agents to be delivered attached to a polymeric molecule, which acts as a carrier. The agents are thus disposed on the polymer backbone, and are carried into the target cell together with the polymer.

The agents may be conjugated (i.e., coupled, fused, mixed, combined, or otherwise joined) to a polymer. The coupling between the agent and the polymer may be permanent or transient, and may involve covalent or non-covalent interactions (including ionic interactions, hydrophobic forces, Van der Waals interactions, etc). The exact mode of coupling is not important, so long as the agent is taken into a target cell substantially together with the polymer. For simplicity, the entity comprising the agent attached to the polymer carrier is referred to here as a “polymer-agent conjugate.”

Any suitable polymer, for example, a natural or synthetic polymer, may be used. Preferably the carrier polymer is a synthetic polymer such as PEG. More preferably, the carrier polymer is a biologically inert molecule. Particular examples of polymers include polyethylene glycol (PEG), N-(2-hydroxypropyl) methacrylamide (HPMA) copolymers, polyamidoamine (PAMAM) dendrimers, HEMA, linear polyamidoamine polymers etc.

Any suitable linker for attaching the agent to the polymer may be used. Preferably, the linker is a biodegradable linker. Use of biodegradable linkers enables controlled release of the agent on exposure to the extracellular or intracellular environment. High molecular weight macromolecules are unable to diffuse passively into cells, and are instead engulfed as membrane-encircled vesicles. Once inside the vesicle, intracellular enzymes may act on the polymer-agent conjugate to effect release of the agent. Controlled intracellular release circumvents the toxic side effects associated with many drugs.

Furthermore, agents may be conjugated by methods known in the art to any suitable polymer, and delivered. The agents may comprise any of the molecules referred to as “second agents,” such as polypeptides, nucleic acids, macromolecules, etc, as described in the section above. In particular, the agent may comprise a prodrug.

The ability to choose the starting polymer enables the engineering of polymer-agent conjugates with desirable properties. The molecular weight of the polymer (and thus the polymer-agent conjugate), as well as its charge and hydrophobicity properties, may be precisely tailored. Advantages of using polymer-agent conjugates include economy of manufacture, stability (longer shelf life) and reduction of immunogencity and side effects.

Furthermore, polymer-agent conjugates are especially useful for the targeting of tumor cells because of the enhanced permeability and retention (EPR) effect, in which growing tumors are more ‘leaky’ to circulating macromolecules and large particles, allowing them easy access to the interior of the tumor. Increased accumulation and low toxicity (typically 10-20% of the toxicity of the free agent) are also observed. Use of hyperbranched dendrimers, for example, PAMAM dendrimers, is particularly advantageous in that they enable monodisperse compositions to be made and also flexibility of attachment sites (within the interior or the exterior of the dendrimer).

The pH responsiveness of polymer-agent conjugates, for example, those conjugated to polyamindoamine polymers, may be tailored for particular intracellular environments. This enables the drug to be released only when the polymer therapeutic encounters a particular pH or range of pH, i.e., within a particular intracellular compartment. The polymer agent conjugates may further comprise a targeting means, such as an immunoglobulin or antibody, which directs the polymer-agent conjugate to certain tissues, organs or cells comprising a target, for example, a particular antigen. Other targeting means are described elsewhere in this document, and are also known in the art.

Particular examples of polymer-agent conjugates include “Smancs”, comprising a conjugate of styrene-co-maleic anhydride and the antitumour protein neocarzinostatin, and a conjugate of PEG (poly-ethylene glycol) with L-asparaginase for treatment of leukaemia; PK1 (a conjugate of a HPMA copolymer with the anticancer drug doxorubicin); PK2 (similar to PK1, but furthermore including a galactose group for targeting primary and secondary liver cancer); a conjugate of HPMA copolymer with the anticancer agent captothecin; a conjugate of HPMA copolymer with the anticancer agent paclitaxel; HPMA copolymer-platinate, etc. Any of these polymer-agent conjugates are suitable for co-loading into the transgenic cells of the present invention.

Loading a Cell Delivery Vehicle With One or More Agent-MTS Conjugates

The agent-MTS conjugates according to the invention can be loaded into cells, such as red blood cells, by any suitable means, as described in further detail below. Loading of a cell with more than one agent may be performed such that the agents are loaded individually (in sequence) or together (simultaneously or concurrently). Such co-loading may involve any combination of agent-MTS conjugates.

It will be appreciated that, because of the presence of a membrane translocation sequence, the agent-MTS conjugates are capable of crossing the red blood cell membrane and therefore can “self-load” into a cell with little or no farther assistance. Thus, in one embodiment, the invention includes a method of loading a cell comprising, exposing the cell to an agent-MTS conjugate for a period of time sufficient to enable uptake of the agent—MTS conjugate by the cell. Such a passive loading method, or “auto loading method” does not require energy.

Progress of loading may be monitored by any suitable means. In one embodiment, where cells are loaded with protein/polypeptide or peptide agents, a sample of loaded cells can be obtained at different time periods and the amount of protein/polypeptide/or peptides can be measured using standard protein detection methods (e.g., such as by immunoassays using an antibody which specifically binds the agent). In another embodiment, where the agent is a nucleic acid, standard nucleic acid detection methods can be used, such as a hybridization assay using a probe which specifically binds to the agent.

In a further embodiment, a cell is actively loaded with an agent-MTS conjugate, for example, by using hypotonic dialysis. In one embodiment, active loading is performed by a procedure selected from the group consisting of electroporation, iontophoresis, sonoporation, microinjection, calcium precipitation, membrane intercalation, microparticle bombardment, lipid-mediated transfection, viral infection, osmosis, osmotic pulsing, osmotic shock, diffusion, endocytosis, mechanical perforation/restoration of the plasma membrane by shearing, single-cell injection and combinations thereof.

Sonoporation as a method for loading an agent into a cell is disclosed in, for example, Miller, et al., 1998, Ultrasonics, 36: 947-952, the entirety of which is incorporated by reference herein.

Iontophoresis uses an electrical current to activate and to modulate the diffusion of a charged molecule across a biological membrane, such as the skin, in a manner similar to passive diffusion under a concentration gradient, but at a facilitated rate. In general, iontophoresis technology uses an electrical potential or current across a semipermeable barrier. By way of example, delivery of heparin molecules to patients has been shown using iontophoresis, a technique which uses low current (i.e., D.C.) to drive charged species into the arterial wall. The iontophoresis technology and references relating thereto is disclosed in WO 97/49450, the entirety of which is incorporated by reference herein.

In a preferred embodiment of the invention, loading takes place by way of hypotonic dialysis, for example using a dialysis device. Dialysis devices work on the principle of osmotic shock, whereby loading of an agent into a cell, such as a red blood cell, is facilitated by the induction of sequential hypotonicity and recovery of isotonicity. The term “osmotic shock” is intended herein to be synonymous with the term “hypotonic dialysis” or “hypoosmotic dialysis.”An exemplary osmotic shock/hypotonic dialysis method is described in Eichler, et al., 1986, Res. Exp. Med. 186: 407-412, the entirety of which is incorporated by reference herein.

A preferred osmotic shock/hypotonic dialysis method is based on the method described in Eichler, et al., 1986, Res. Exp. Med., 186: 407-412. This preferred method is as follows. Washed red blood cells are suspended in 1 ml of PBS (150 mM NaCl, 5 mM K₂HPO₄/KH₂PO₄ pH 7.4) to obtain a hematocrit of approximately 60%. The suspension is placed in dialysis tubing (molecular weight cut-off 12-14,000; Spectra-Por; prepared as outlined below) and swelling of cells obtained by dialysis against 100 ml of 5 mM K₂HPO₄/KH₂PO₄, pH 7.4 for 90 minutes at 4° C. Resealing is achieved by subsequent dialysis for 15 minutes at 37° C. against 100 ml of PBS containing 10 mM glucose. Cells are then washed in ice cold PBS containing 10 mM glucose using centrifugation.

Other osmotic shock procedures include the method described in U.S. Pat. No. 4,478,824, the entirety of which is incorporated by reference herein. That method involves incubating a packed red blood cell fraction in a solution containing a compound (such as dimethyl sulphoxide (DMSO) or glycerol) which readily diffuses into and out of cells, rapidly creating a transmembrane osmotic gradient by diluting the suspension of red blood cell in the solution with a near-isotonic aqueous medium. This medium contains an anionic agent to be introduced (such as inosine monophosphate or a phosphorylated inositol, for example inositol hexaphosphate) which may be an allosteric effector of haemoglobin, thereby causing diffusion of water into the cells with consequent swelling thereof and increase in permeability of the outer membranes of the cells. This increase in permeability is maintained for a period of time sufficient only to permit transport of the anionic agent into the cells and diffusion of the readily-diffusing compound out of the cells. This method is not the method of choice where the desired agent to be loaded into cells is not anionic, or is anionic or polyanionic, but is not present in the near-isotonic aqueous medium in sufficient concentration to cause the needed increase in cell permeability without cell destruction.

U.S. Pat. No. 4,931,276 and WO 91/16080 also disclose methods of loading red blood cells with selected agents using an osmotic shock technique. Therefore, these techniques can be used to enable loading of red blood cells in the present invention.

An alternative osmotic shock procedure is described in U.S. Pat. No. 4,931,276 which is incorporated herein by reference.

Alternatively, loading may be carried out by a microparticle bombardment procedure. Microparticle bombardment entails coating gold particles with the agent to be loaded, dusting the particles onto a 22 calibre bullet, and firing the bullet into a restraining shield made of a bulletproof material and having a hole smaller than the diameter of the bullet, such that the gold particles continue in motion toward cells in vitro and, upon contacting these cells, perforate them and deliver the payload (e.g., the agent-MTS conjugate) to the cell cytoplasm.

It will be appreciated by one skilled in the art that combinations of methods may be used to facilitate the loading of a cell with agents of interest according to the invention. Likewise, it will be appreciated that a first and second agent, may be loaded concurrently or sequentially, in either order, into a red blood cell in any method of the present invention. It will be understood that the invention is not limited to loading of a first and/or second agent; third and subsequent agents may also be loaded in the same manner as described here.

It will be appreciated by one skilled in the art that combinations of methods may be used to facilitate the loading of a red blood cell with agents of interest according to the invention. Likewise, it will be appreciated that a first and second agent, may be loaded concurrently or sequentially, in either order, into a red blood cell in any method of the present invention.

The concentration of agent used in the loading procedure may need to be optimized using routine techniques. Preferably loading takes place over a period of at least 30 minutes, more preferably about 90 minutes.

While in some embodiments, agent-MTS conjugates are loaded into a cell, in other embodiments, a nucleic acid encoding an agent-MTS conjugate is loaded into the cell, either passively, or actively, as described above. In this embodiment, the fusion protein expressed by these sequences is loaded in the cell through the translation of mRNA expressed by the nucleic acid within the cell.

Sensitisation

In a highly preferred embodiment of the invention, loaded cells are sensitised to render them more susceptible to disruption by a stimulus than unsensitised cells, such that the delivery vehicle will release its contents at a target site while surrounding cells remain substantially unaffected (e.g., less than 10% of the surrounding cells are disrupted).

The invention therefore encompasses the use of sensitising agents and/or processes to increase the susceptibility of cells, such as red blood cells, to disruption using energy such as ultrasound or light energy. The vehicles of the invention are preferably capable of being selectively disrupted at a target site by exposure to a stimulus, for example, laser light or ultrasound. Preferred sensitisation procedures, such as electrosensitisation, are set forth in International Patent Application Number PCT/GB00/02848 and in U.S. Patent application Ser. No. ______, filed Feb. 8, 2001, (Attorney Docket No. 11067/2042), the entireties of which are incorporated herein.

As will be apparent to one of skill in the art, any one of the above described loading techniques can be used prior to, simultaneously with, separate from, or in sequence with, the sensitisation procedure. For example, U.S. Pat. No. 4,224,313, the entirety of which is incorporated by reference herein, discloses a process for preparing a mass of loaded red blood cells suspended in a solution by increasing the permeability of the cell membranes by osmotic pressure, or an electric field, or both, loading agents by passage from a solution through the membranes of increased permeability, restoring the original permeability of the cells' membranes by sealing the membranes using a regeneration effect, and separating the cells from the solution in which they are suspended. In that procedure, the agents in solution which are to be loaded include i) a pharmaceutical substance which reacts chemically or physically with substances in the extracellular milieu and which, when loaded into the cell, would prematurely destroy the cell membranes, and ii) at least one blood-compatible sugar and protein capable of providing hydrogen bridge bonding- or of entering into covalent bonds with the pharmaceutical substance, thereby inhibiting the reaction of the pharmaceutical substance with the cell membranes.

Preferably, the sensitisation step comprises an electrosensitisation procedure as described further in the Examples below. The efficiency of sensitisation for given electrical parameters may vary depending on the cell density and it may therefore be necessary to perform a titration of cell density and or electrical parameters to establish the optimum concentration. By way of guidance, cells sensitised at a density of about 6-8×10⁸ cells/ml have been found to have good sensitivity to ultrasound.

Generally, where present, the sensitisation step(s) and the loading step(s) are temporally separated. For example, cells are typically allowed to rest in buffer, such as PBS/Mg/glucose buffer, for at least 30 minutes, preferably at least 60 minutes, after a pre-sensitisation step to allow the cells to recover prior to loading or further sensitisation steps. It may be desirable to allow cells to rest for several hours, such as overnight, after the loading step. However, where passive loading is used, the sensitisation step may be effectively carried out at the same time as the agent is being loaded.

Electrosensitisation

The delivery vehicles (e.g., red blood cells) of the present invention may be sensitised to ultrasound or other sources of energy by the use of an electric field (“electrosensitisation”). Electrosensitisation may also be used as a means of pre-sensitising red blood cells.

The term “electrosensitisation” encompasses the destabilisation of cells without causing fatal damage to the cells. According to this method, a momentary exposure of a cell to one or more pulses at high electric field strength results in membrane destabilisation. The strength of the electric field is adjusted up or down depending upon the resilience or fragility, respectively, of the cells being loaded and the ionic strength of the medium in which the cells are suspended.

Electrosensitisation typically occurs in the absence of the agent to be loaded into the cell. Electroporation, which facilitates passage of agents into the cell, occurs in the present of an exogenous agent to be loaded, and is well known in the art.

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both supplied by the BTX Division of Genetronics, Inc (see U.S. Pat. No. 5,869,326).

These known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1 kV/cm, of about 100 μs duration. Such a pulse may be generated, for example, in known applications of the Electro Square Porator T820.

Electrosensitisation may be performed in a manner substantially identical to the procedure followed for electroporation, with the exception that the electric field is delivered in the absence of an exogenous agent of interest, as set forth below, and may be carried out at different electric field strengths (and other parameters) from those required for electroporation. For example, lower field strengths may be used for electrosensitisation.

Preferably, the electric field has a strength of from about 0.1 kV/cm to about 10 kV/cm under in vitro conditions, more preferably from about 1.5 kV/cm to about 4.0 kV/cm under in vitro conditions. Most preferably, the electric field strength is about 3.625 kV/cm under in vitro conditions.

Preferably the electric field has a strength of from about 0.1 kV/cm to about 10 kV/cm under in vivo conditions (see WO 97/49450). More preferably, the electric field strength is about 3.625 kV/cm under in vitro conditions.

Preferably the application of the electric field is in the form of multiple pulses such as double pulses of the same strength and capacitance or sequential pulses of varying strength and/or capacitance. A preferred type of sequential pulsing comprises delivering a pulse of less than 1.5 kV/cm and a capacitance of greater than 5 μF, followed by a pulse of greater than 2.5 kV/cm and a capacitance of less than 2° F., followed by another pulse of less than 1.5 kV/cm and a capacitance of greater than 51F. A particular example is 0.75 kV/cm, 10 μF; 3.625 kV/cm, 1 μF and 0.75 kV/cm, 10 μF.

Preferably the electric pulse is delivered as a waveform selected from an exponential wave form, a square wave form and a modulated wave form.

Other electroporation procedures and methods employing electroporation devices are widely used in cell culture, and appropriate instrumentation, including the use of flow cell technology, is well known in the art. These procedures and methods may be adapted to perform electrosensitisation on a red blood cell.

In a particularly preferred embodiment, the following electrosensitisation protocol is used. Cells are suspended in PBS to yield concentrations of about 6-8×10⁸ cells/ml and 0.8 ml aliquots are dispensed into sterile electroporation cuvettes (0.4 cm electrode gap) and retained on ice for 10 min. Cells are then exposed to an sensitisation strategy involving delivery of two electric pulses (field strength=3.625 kV/cm at a capacitance of 1 μF) using a BioRad Gene Pulser apparatus. Cells are immediately washed with PBS containing MgCl₂ (4 mM) (PBS/Mg) and retained at room temperature for at least 30 min in the PBS/Mg buffer at a concentration of 7 ×10⁸ cells/ml to facilitate re-sealing. Optionally, cells are subsequently washed and suspended at a concentration of 7×10⁸ cells/ml in PBS/Mg containing 10 mM glucose (PBS/Mg/glucose) for at least 1 hour.

Pre-Sensitisation

The delivery vehicles of the invention may also be subjected to a pre-sensitising step to increase the efficiency of the loading process. A preferred pre-sensitising step involves applying an electric field to the cells, as described in International Patent Application Number PCT/GB00/03056, and in U.S. patent application Ser. No. ______, filed Feb. 8, 2001, (Attorney Docket No. 11067/2042), the entirety of which is incorporated by reference herein.

Cells can be pre-sensitised whether loaded by an active or passive loading procedure and multiple rounds of loading and/or pre-sensitisation may be performed.

Pre-sensitisation may take the form of an electrosensitisation step, as described further in the Examples below. Alternatively, or in addition, pre-sensitisation may be effected by, for example the use of ultrasound, electromagnetic radiation such as microwaves, radio waves, gamma rays and X-rays. In addition, chemical agents such as DMSO and pyrrolidinone nay be used. Furthermore, cells may be exposed to thermal energy to pre-sensitise them. This may be achieved by raising the temperature of the cells by conventional means, by heat shock, or by the use of microwave irradiation. In general, any method which allows pores to be formed on the surface membrane of a cell, such as a red blood cell, is a suitable candidate for use as a pre-sensitisation step. Pre-sensitisation methods are described further in U.S. patent application Ser. No. ______, filed Feb. 8, 2001 (Attorney Docket No. 11067/2042), the entirety of which is incorporated by reference herein.

Where a pre-sensitisation step is undertaken, cells may be loaded either after the pre-sensitisation procedure or after one or more sensitisation procedures, and preferably after the cells have rested. In this embodiment, the loading may be performed by any desired technique. Thus, a pre-sensitised and loaded cell may be sensitised. Furthermore, a pre-sensitised and subsequently sensitised cell may be loaded.

Pre-Sensitisation Using Ultrasound

Where a pre-sensitisation step is present, this typically involves electrosensitisation (described in detail below); however, in one embodiment, ultrasound may also be used to pre-sensitise red blood cells. Such use of ultrasound is also referred to herein as “sonoporation”. Exposure of red blood cells to ultrasound is believed to result in non-destructive and transient membrane poration (see, e.g., Miller et al, 1998, Ultrasonics 36, 947-952, the entirety of which is incorporated by reference herein).

The lower frequency limit of the ultrasonic spectrum may generally be taken as about 20 kHz. Most diagnostic applications of ultrasound employ frequencies in the range 1 and 15 MHz (from Ultrasonics in Clinical Diagnosis. Edited by PNT Wells, 2nd. Edition, Publ. Churchill Livingstone [Edinburgh, London & NY, 1977], the entirety of which is incorporated by reference herein).

Ultrasound has been used in both diagnostic and therapeutic applications. When used as a diagnostic tool (“diagnostic ultrasound”), ultrasound is typically used in an energy density range of up to about 100 mW/cm² (FDA recommendation), although energy densities of up to 750 mW/cm² have been used. In physiotherapy, ultrasound is typically used as an energy source in a range up to about 3 to 4 W/cm² (WHO recommendation). In other therapeutic applications, higher intensities of ultrasound may be employed, for example, HIFU at 100 W/cm² up to 1 kW/cm² (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz, et al., 1998, Journal of Magnetic Resonance Imaging, 8(1):136-142. Another form of focused ultrasound is high intensity focused ultrasound (HIFU) which is reviewed by Moussatov, et al., 1998, Ultrasonics, 36(8): 893-900 and TranHuuHue, et al., 1997, Acustica, 83(6): 1103-1106.

Preferably, cells are pre-sensitised by exposure to ultrasound that has an energy density in the therapeutic range. In a highly preferred embodiment, treatment is at 2.5 W/cm² for 5 min using a 1 MHz ultrasound head. This combination is however not intended to be limiting. Indeed, various combinations of frequency, energy density and exposure time may be used to pre-sensitise cells so that their loading efficiency is increased. In a preferred embodiment, the cells which are pre-sensitised are red blood cells.

Although the purpose of the pre-sensitisation step is to enhance the loading of the agent, an increase in sensitivity to lysis (for example, ultrasound mediated lysis) may also be achieved. Where more than one sensitisation step is involved, additional sensitisation steps may be performed at any stage in the process after the pre-sensitisation step. Thus, a second sensitisation step may be carried out either after the pre-sensitisation step but prior to loading, or after loading. Further sensitisation steps may be performed as required.

Delivery of Loaded Cells to a Target Site

In one embodiment, cells are delivered to a target site, by introducing the cells intravenously into the body of a mammal, such as a human being. Preferably, the cells used as delivery vehicles are red blood cells, and most preferably, the cells are human red blood cells (e.g., autologous red blood cells). In one embodiment, cells are introduced into the body of a mammal using a hollow needle, such as a hypodermic needle or cannula, inserted through the wall of a blood vessel (e.g., a vein or artery) and the cell is either injected using applied pressure or allowed to diffuse or otherwise migrate into the blood vessel. It is understood that the diameter of the needle is sufficiently large and the pressure sufficiently light to avoid damage of the cell by shear forces. Preferably, introduction of a cell, such as a red blood cell, is performed by intra-arterial or intravenous injection. Methods of blood cell transfusion are well known in the art.

In one embodiment, the delivery vehicle is allowed to reach a target site and is disrupted upon arrival at a target site by a stimulus, causing release of the agent-MTS conjugates loaded therein. Because of the MTS portion of the conjugates, the agents are capable of translocating across the membrane of cells at the target site, to effectuate the bioeffector activity of the agents within cells at the target site.

The agents which are loaded into a cell may be released from the cell and into its surroundings, i.e., at or into the target site, tissue or cell, by the application of ultrasound directed at a target site, tissue and/or cell. Furthermore, the agent may be delivered to the target site by application of ultrasound to vessels, for example, blood vessels, feeding the target site.

Disruption of the delivery vehicle may be focused in a single tissue, such as by providing a source of ultrasonic energy (e.g., an ultrasonic probe) in proximity to the tissue via a medical access device (e.g., such as a catheter or endoscope). In this embodiment, placement of the source of energy can be facilitated by using an optical system (e.g., an optical fiber in communication with a light source and one or more light directing elements) in conjunction with the source of energy.

In an alternative embodiment, the entire body of the organism may be exposed to the stimulus (e.g., ultrasonic energy). Similarly, the energy levels used may release the contents of substantially all of the delivery vehicles, or only part of these. In the second case, repeated applications of energy may be used to provide additional therapeutic doses of an agent.

The present invention is useful for the delivery of therapeutic or diagnostic agents to specific sites in vertebrate organisms, without the problems associated with agents being unable to cross the cell membrane. The ability to selectively disrupt delivery vehicles according to the invention permits the person skilled in the art to achieve release of the contents of the delivery vehicles at any desired site to which the stimulus required may be directed.

Selective Release Using Ultrasound

Preferably, a combination of diagnostic ultrasound and a therapeutic ultrasound is employed to effect selective release. This combination is not intended to be limiting, however, and the skilled artisan will appreciate that any variety of combinations of ultrasound may be used. Additionally, the energy density, frequency of ultrasound, and period of exposure may be varied. What is important is that the application of ultrasound is able to selectively disrupt the sensitised cells to effect release of agent, without substantially disrupting or damaging endogenous cells, i.e., non-loaded cells.

Preferably the ultrasound is applied to a target cell or target tissue with sufficient strength to disrupt loaded and sensitised cells but without damaging the target tissue or surrounding tissues. In this context, the term “damage or damaging” does not include a transient permeabilisation of the target site by the ultrasound energy source. Such a permeabilisation may facilitate uptake of the released payload at the target site.

Preferably the exposure to an ultrasound energy source is at a power density of from about 0.05 to about 100 Wcm⁻². Even more preferably, the exposure to an ultrasound energy source is at a power density of from about 1 to about 15 Wcm⁻².

Preferably the exposure to an ultrasound energy source is at a frequency of from about 0.015 to about 10.0 MHz. More preferably the exposure to an ultrasound energy source is at a frequency of from about 0.02 to about 6.0 MHz.

Preferably the exposure is for periods of from about 10 milliseconds to about 60 minutes. More preferably the exposure is for periods of from about 1 second to about 5 minutes. Depending on the amount of agent which it is desired to release, however, the exposure may be for a longer duration, for example, for 15 minutes.

Particularly preferably, a patient is exposed to an ultrasound energy source at an acoustic power density of from about 0.05 Wcm⁻² to about 10 Wcm⁻² with a frequency ranging from about 0.015 to about 10 MHz (see WO 98/52609). However, alternatives are also possible, for example, exposure to an ultrasound energy source at an acoustic power density of above 100 Wcm⁻², but for reduced periods of time, for example, 1000 Wcm⁻² for periods in the millisecond range or less.

Use of ultrasound is advantageous because, like light, it can be focused accurately on a target. Moreover, ultrasound is advantageous as it can be focussed more deeply into tissues unlike light. It is therefore better suited to whole-tissue penetration (such as but not limited to a lobe of the liver) or whole organ (such as but not limited to the entire liver or an entire muscle, such as the heart) delivery of agents according to the present invention. In addition, ultrasound may induce a transient permeabilisation of the target site so that uptake of a released payload is facilitated at the target site. Another important advantage is that ultrasound is a non-invasive stimulus which is used in a wide variety of diagnostic and therapeutic applications. By way of example, ultrasound is well known in medical imaging techniques and, additionally in orthopaedic therapy. Furthermore, instruments suitable for the application of ultrasound to a subject vertebrate are widely available and their use is well known in the art.

In methods of the invention, release of the agent is effected by exposure of cells, such as red blood cells, either in vitro or ex-vivo to an effective amount of a diagnostic ultrasound energy source or a therapeutic ultrasound energy source as described in U.S. Pat. No. 5,558,092 and WO 94/28873, the entireties of which are incorporated by reference herein. The agent, which is released from a red blood cell for use in the present invention may be referred to as the “payload” of that cell.

Preferably the agent is released from the red blood cell by treatment of a target site, tissue or cell with ultrasound.

The selective release of the agent at the target site can be determined by observing a) the amount which has been released at the target site, tissue or cell and b) its effect on the target site, tissue or cell, the latter determining whether its delivery should increase, decrease or be discontinued.

Expression of Transgenes Encoding Agent-MTS Conjugates

In some embodiments, the agent-MTS conjugate comprises a fusion protein encoded by a transgene expressed in red blood cells (RBCs). This aspect of the invention is described in more detail in co-pending British Patent Application No. 0101469.5, the entirety of which is incorporated by reference herein. In this embodiment, the transgene is driven by, or operably linked to, a promoter that is specific for an erythroid cell lineage, and most preferably, a reticulocyte cell lineage.

Reticulocytes are immature RBCs which have extruded their nucleus, but retain a large amount of RNA, and thus display a grainy basophilic staining pattern in hematoxylin and eosin stained preparations. Circulating reticulocytes, which make up approximately 1% of circulating blood cells are transient blood cells; after leaving the bone marrow, reticulocytes retain their RNA and thus their protein synthetic ability for approximately 24 hours, before full maturation into essentially mRNA-free erythrocytes. During its life cycle in circulating blood, reticulocytes, by virtue of their RNA content, continue to produce haemoglobin and thus continue to translate mRNAs, endogenous or recombinant, derived from genes which possess erythrocyte-specific promoters. Therefore, the fusion proteins described above, driven by the erythrocyte promoters described below, will be expressed in virtually all circulating RBCs by virtue of transgene synthesis in reticulocytes prior to their maturation to mature RBCs.

Any promoter known to be active in cells of the erythrocytic lineage may be used to direct the expression of a polypeptide in the methods of the invention. However, examples of promoters that direct high level expression of erythroid-specific genes include the globin gene promoters. Haemoglobin is expressed in a tissue-specific manner in RBCs, where it accounts for about 95% of total cellular protein. Globin gene promoters include those for the I, J (β globin), L, M and N globin genes. Particularly preferred among these is the human β globin promoter, which is most active in adults.

ε, γ^{G}, γ^{A}, δ and β Globin Gene Promoters

Human β globin (also known as J globin) genes are found in a cluster on chromosome 11, comprising about 50 kb of DNA that also includes one embryonic gene encoding ε globin (also known as M globin), two fetal genes encoding K globins γ_{G}, γ^{A} (also known as G and A globins), and two adult genes encoding δ and β globin (also known as L and J globin), in that order (Fritsch et al., 1980, Cell, 19: 959-972). It has been found that DNA sequences both upstream and downstream of the β globin translation initiation site are involved in the regulation of β globin gene expression (Wright et al., 1984, Cell, 38: 263). In particular, a series of four DNAse I super hypersensitive sites (now referred to as the locus control region, or LCR) located about 50 kilobases upstream of the human β globin gene are extremely important in eliciting properly regulated β globin-locus expression (see, e.g., Tuan et al., 1985, Proc. Natl. Acad. Sci. U.S.A., 83: 1359-1363; PCT Patent Application WO 89/01517; Behringer et al., 1989, Science, 245: 971-973; Enver et al., 1989, Proc. Natl. Acad. Sci. U.S.A., 86: 7033-7037; Hanscombe et al., 1989, Genes Dev., 3: 1572-1581; Van Assendelft et al., 1989, Cell, 56: 967-977; and Grosveld et al., 1987, Cell 51: 975-985, the entireties of which are incorporated by reference herein). Thus, in a highly preferred embodiment of the invention, the transgene is operably linked to, or its expression is regulated from, a globin LCR.

Expression systems, including expression vectors, useful for erythroid expression are described in detail in U.S. Pat. No. 5,538,885 and GB 2251622, the entireties of which are incorporated by reference herein. Such vectors comprise a promoter, a DNA sequence which codes for a desired polypeptide (e.g., agent-MTS conjugates) and a dominant control region. Preferably, the dominant control region comprises a micro locus which comprises a 6.5 kb fragment obtained by ligating the fragments: 2.1 kb XbaI-XbaI; 1.9 kb HindIII-HindIII; 1.5 kb KpnI-BgIII; and 1.1 kb partial SacI; from the β-globin gene. As used herein the term “dominant control region” (or “DCR”) means a sequence of DNA capable of conferring upon a linked gene expression system the property of host cell-type restricted, integration site independent, copy number dependent, expression when integrated into the genome of a host compatible with the dominant control region. The dominant control region retains this property when fully integrated within the chromosome of a host cell; and the ability to direct efficient host cell-type restricted expression is retained even when fully integrated in a heterologous background such as a different part of the homologous chromosome or even a different chromosome.

A method for making a desired peptide in transgenic animals is described in U.S. Pat. No. 5,627,268, the entirety of which is incorporated by reference herein. A transgenic animal is engineered to comprise an artificial gene, which is controlled by globin locus control region (LCR) and which encodes a fusion protein. In the fusion protein, the desired peptide is linked via a cleavable peptide bond to a globin polypeptide. The erythrocytes of the transgenic animal express the fusion protein which is incorporated into hemoglobin produced by the host cell. The desired peptide can be obtained from a hemolysate of the red cells of the transgenic animals by cleavage of the linking bond and separation of the peptide away from globin portions. Production of recombinant haemoglobin is described in U.S. Pat. No. 5,821,351, the entirety of which is incorporated by reference herein.

Other promoters useful in the method of the invention include the promoter of the Erythroid-specific GATA-1 transcription factor gene or a heterologous construct comprising the enhancer from the GATA-1 transcription factor gene (Grande et al., 1999, Blood, 93: 3276). Other alternatives include but are not limited to the NF-E2 proximal IB promoter (Moroni et al., 2000, JBC, 275: 10567) and the B19 p6 promoter with or without an erythrocyte-specific enhancer element (Kurpad et al, 1999, J. Hematother. Stem. Cell. Res., 8: 585). The skilled person will appreciate that any suitable promoter may be used, so long as it directs expression of the desired polypeptide at an appropriate level in the red blood cell.

Transgenic Animals

In one embodiment of the invention, the delivery vehicle comprises a cell which is produced by a transgenic animal. A transgenic animal is a non-human animal containing at least one foreign gene, called a transgene, which is part of its genetic material. Preferably, the transgene is contained in the animal's germ line such that it can be transmitted to the animal's offspring. In relation to the present invention, transgenic animals are useful for producing RBCs comprising polypeptides, in particular therapeutic polypeptides. A number of techniques may be used to introduce the transgene into an animal's genetic material, including, but not limited to, microinjection of the transgene into pronuclei of fertilized eggs and manipulation of embryonic stem cells (U.S. Pat. No. 4,873,191 by Wagner and Hoppe; Palmiter and Brinster, 1986, Ann. Rev. Genet., 20:465-499; French Patent Application 2593827 published Aug. 7, 1987). Transgenic animals may carry the transgene in all their cells or may be genetically mosaic.

According to the method of conventional transgenesis, additional copies of normal or modified genes are injected into the male pronucleus of the zygote and become integrated into the genomic DNA of the recipient mouse. The transgene is transmitted in a Mendelian manner in established transgenic strains.

Constructs useful for creating transgenic animals useful according to the invention comprise genes encoding therapeutic molecules (i.e., agents), preferably under the control of nucleic acid sequences directing their expression in cells of the erythroid lineage. Alternatively, therapeutic molecules encoding constructs may be under the control of their native promoters, or inducibly regulated. A transgenic animal expressing one transgene can be crossed to a second transgenic animal expressing second transgene such that their offspring will carry both transgenes.

Although the majority of studies have involved transgenic mice, other species of transgenic animal have also been produced, such as rabbits, sheep, pigs (Hammer et al., 1985, Nature, 315: 680-683; U.S. Pat. No. 5,922,854; U.S. Pat. No. 6,030,833) and chickens (Salter et al., 1987, Virology, 157: 236-240). While the transgenic animals described in the present invention are not limited to swine, the description which follows details the methodology for transgene expression in larger animals, such as swine, but may be adapted for smaller animals as need requires. Transgenic monkeys have also been described in Chan et al., Mol. Reprod. Dev., 2000 Jun, 56(2 Suppl): 325-8. Transgenic animals are currently being developed to serve as bioreactors for the production of useful pharmaceutical compounds (Van Brunt, 1988, Bio/Technology 6: 1149-1154; Wilmut et al., 1988, New Scientist, (July 7 issue) pp. 56-59).

Methods of expressing recombinant protein via transgenic livestock have an important theoretical advantage over protein production in recombinant bacteria and yeast; namely, the ability to produce large, complex proteins in which post-translational modifications, including glycosylation, phosphorylation, subunit assembly, etc. are critical for the activity of the molecule.

In particular, the present invention includes, but is not limited to, recombinant swine RBCs expressing agent-MTS fusion polypeptides. RBCs containing the agent-MTS fusion polypeptide may be prepared by introducing a recombinant nucleic acid molecule which encodes said agent-MTS fusion polypeptide into a tissue, such as bone marrow cells, using known transformation techniques. These transformation techniques include transfection and infection by retroviruses carrying either a marker gene or a drug resistance gene. See, for example, Current Protocols in Molecular Biology, Ausubel et al, eds., John Wiley and Sons, New York (1987) and Friedmann, 1989, Science, 244: 1275-1281. A tissue containing a recombinant nucleic acid molecule of the present invention may then be reintroduced into an animal using reconstitution techniques (see, for example, Dick et al., 1985, Cell, 42: 71).

The recombinant constructs described here may be used to produce a transgenic animal by any method known in the art, including, but not limited to, microinjection, embryonic stem (ES) cell manipulation, electroporation, cell gun, transfection, transduction, retroviral infection, etc. Transgenic animals of the present invention can be produced by introducing transgenes into the germline of the animal, particularly into the genome of bone marrow cells, e.g. hematopoietic cells. Embryonal target cells at various developmental stages can be used to introduce the human transgene construct. As is generally understood in the art, different methods are used to introduce the transgene depending on the stage of development of the embryonal target cell.

One technique for transgenically altering an animal is to microinject a recombinant nucleic acid molecule into the male pronucleus of a fertilized egg so as to cause 1 or more copies of the recombinant nucleic acid molecule to be retained in the cells of the developing animal. The recombinant nucleic acid molecule of interest is isolated in a linear form with most of the sequences used for replication in bacteria removed. Linearization and removal of excess vector sequences results in a greater efficiency in production of transgenic mammals. See for example, Brinster et al., 1985, PNAS, 82: 4438-4442.

In general, the zygote is the best target for micro-injection. In the swine, the male pronucleus reaches a size which allows reproducible injection of DNA solutions by standard microinjection techniques. Moreover, the use of zygotes as a target for gene transfer has a major advantage in that, in most cases, the injected DNA will be incorporated into the host genome before the first cleavage. Usually up to 40 percent of the animals developing from the injected eggs contain at least 1 copy of the recombinant nucleic acid molecule in their tissues. These transgenic animals will generally transmit the gene through the germ line to the next generation. The progeny of the transgenically manipulated embryos may be tested for the presence of the construct by Southern blot analysis of a segment of tissue. Typically, a small part of the tail is used for this purpose.

The stable integration of the recombinant nucleic acid molecule into the genome of transgenic embryos allows permanent transgenic mammal lines carrying the recombinant nucleic acid molecule to be established. Alternative methods for producing a mammal containing a recombinant nucleic acid molecule of the present invention include infection of fertilized eggs, embryo-derived stem cells, to potent embryonal carcinoma (EC) cells, or early cleavage embryos with viral expression vectors containing the recombinant nucleic acid molecule. (See for example, Palmiter et al., 1986, Ann.Rev. Genet. 20: 465-499 and Capecchi, 1989, Science, 244: 1288-1292.)

Retroviral infection can also be used to introduce transgene into an animal, including swine. The developing embryo can be cultured in vitro to the blastocyst stage. During this time, the blastomeres can be targets for retroviral infection (Jaenich, 1976, PNAS, 73: 1260-1264). Efficient infection of the blastomeres is obtained by enzymatic treatment to remove the zona pellucida (Hogan et al, 1986, in Manipulating the Mouse Embryo, Cold Spring Harbor Laboratory Press, Cold Spring Harbor, N.Y.). The viral vector system used to introduce the transgene is typically a replication-defective retrovirus carrying the transgene (Jahner et al., 1985, PNAS, 82: 6927-6931; Van der Putten et al., 1985, PNAS, 82: 6148-6152).

Transfection can be obtained by culturing the blastomeres on a monolayer of virus-producing cells (Van der Putten, supra; Stewart et al., 1987, EMBO J., 6: 383-388). Alternatively, infection can be performed at a later stage. Virus or virus-producing cells can be injected into the blastocoele (Jahner et al., 1982, Nature, 298: 623.628). Most of the founders will be mosaic for the transgene since incorporation typically occurs only in a subset of the cells which formed the transgenic swine. Further, the founder may contain various retroviral insertions of the transgene at different positions in the genome which generally will segregate in the offspring. In addition, it is also possible to introduce transgenes into the germ line, albeit with low efficiency, by intrauterine retroviral infection of the mid-gestation embryo (Jahner et al., supra).

A third approach, which may be useful in the construction of tansgenic animals, would target transgene introduction into an embryonal stem cell (ES). ES cells are obtained from pre-implantation embryos cultured in vitro and fused with embryos (Evans et al., 1981, Nature, 292: 154-156; Bradley et al., 1984, Nature, 309: 255-258; Gossler et al., 1986, PNAS, 83: 9065-9069; and Robertson et al., 1986, Nature, 322: 445-448). Transgenes might be efficiently introduced into the ES cells by DNA transfection or by retrovirus-mediated transduction. Such transformed ES cells could thereafter be combined with blastocysts from the same species. The ES cells could be used thereafter to colonize the embryo and contribute to the germ line of the resulting chimeric animal. For review, see Jaenisch, 1988, Science, 240: 1468-1474.

Introduction of the recombinant gene at the fertilized oocyte stage ensures that the gene sequence will be present in all of the germ cells and somatic cells of the transgenic “founder” animal. As used herein, founder (abbreviated “F”) means the animal into which the recombinant gene is introduced at the one cell embryo stage. The presence of the recombinant gene sequence in the germ cells of the transgenic founder animal in turn means that approximately half of the founder animal's descendants will carry the activated recombinant gene sequence in all of their germ cells and somatic cells. Introduction of the recombinant gene sequence at a later embryonic stage might result in the gene's absence from some somatic cells of the founder animal, but the descendants of such an animal that inherit the gene will carry the activated recombinant gene in all of their germ cells and somatic cells.

Microinjection Of Swine Oocytes

In preferred embodiments the transgenic animals of the present invention, including but not limited to swine are produced by: i) microinjecting a recombinant nucleic acid molecule encoding a polypeptide into a fertilized egg to produce a genetically altered egg; ii) implanting the genetically altered egg into a host female animal of the same species; iii) maintaining the host female for a time period equal to a substantial portion of the gestation period of said animal fetus; iv) harvesting a transgenic animal having at least one cell that has developed from the genetically altered mammalian egg, which expresses a gene which encodes a polypeptide.

In general, the use of microinjection protocols in transgenic animal production is typically divided into four main phases: (a) preparation of the animals; (b) recovery and maintenance in vitro of one or two-celled embryos; (c) microinjection of the embryos and (d) reimplantation of embryos into recipient females. The methods used for producing transgenic livestock, particularly swine, do not differ in principle from those used to produce transgenic mice. Compare, for example, Gordon et al., 1983, Methods in Enzymology, 101: 411, and Gordon et al., 1980, PNAS, 77: 7380 concerning, generally, transgenic mice with Hammer et al., 1985, Nature, 315: 680, Hammer et al., 1986, J Anim. Sci., 63: 269-278, Wall et al., 1985, Biol. Reprod., 32: 645-651, Pursel et al., 1989, Science, 244: 1281-1288, Vize et al., 1988, J. Cell Science, 90: 295-300, Muller et al., 1992, Gene, 121: 263-270, and Velander et al., (1992) PNAS, 89: 12003-12007, each of which teach techniques for generating transgenic swine. See also, PCT Publication WO 90/03432, and PCT Publication WO 92/22646, and references cited therein.

One step of the preparatory phase comprises synchronizing the estrus cycle of at least the donor females, and inducing superovulation in the donor females prior to mating. Superovulation typically involves administering drugs at an appropriate stage of the estrus cycle to stimulate follicular development, followed by treatment with drugs to synchronize estrus and initiate ovulation. As described in the example below, a pregnant female animal's serum is typically used to mimic the follicle-stimulating hormone (FSH) in combination with human chorionic gonadotropin (hCG) to mimic luteinizing hormone (LH). The efficient induction of superovulation depends, as is well known, on several variables including the age and weight of the females, and the dose and timing of the gonadotropin administration. See for example, Wall et al., 1985, Biol. Reprod., 32:645 describing superovulation of pigs. Superovulation increases the likelihood that a large number of healthy embryos will be available after mating, and further allows the practitioner to control the timing of experiments.

After mating, one or two-cell fertilized eggs from the superovulated females are harvested for microinjection. A variety of protocols useful in collecting eggs from animals are known. For example, in one approach, oviducts of fertilized superovulated females can be surgically removed and isolated in a buffer solution/culture medium, and fertilized eggs expressed from the isolated oviductal tissues. See, Gordon et al., 1980, PNAS, 77: 7380; and Gordon et al., 1983, Methods in Enzymology, 101: 411. Alternatively, the oviducts can be cannulated and the fertilized eggs can be surgically collected from anesthetized animals by flushing with buffer solution/culture medium, thereby eliminating the need to sacrifice the animal. See Hammer et al., 1985, Nature, 315: 600. The timing of the embryo harvest after mating of the superovulated females can depend on the length of the fertilization process and the time required for adequate enlargement of the pronuclei. This temporal waiting period can range from, for example, up to 48 hours for larger animal species. Fertilized eggs appropriate for microinjection, such as one-cell ova containing pronuclei, or two-cell embryos, can be readily identified under a dissecting microscope.

The equipment and reagents needed for microinjection of the isolated embryos from larger animals are similar to that used for the mouse. See, for example, Gordon et al., 1983, Methods in Enzymology, 101: 411; and Gordon et al., 1980, PNAS, 77: 7380, describing equipment and reagents for microinjecting embryos. Briefly, fertilized eggs are positioned with an egg holder (fabricated from 1 mm glass tubing), which is attached to a micro-manipulator, which is in turn coordinated with a dissecting microscope optionally fitted with differential interference contrast optics. Where visualization of pronuclei is difficult because of optically dense cytoplasmic material, such as is generally the case with swine embryos, centrifugation of the embryos can be carried out without compromising embryo viability. Wall et al., 1985, Biol. Reprod., 32: 645. Centrifugation will usually be necessary in this method.

A recombinant nucleic acid molecule of the present invention is provided, typically in linearized form, by linearizing the recombinant nucleic acid molecule with at least 1 restriction endonuclease, with an end goal being removal of any prokaryotic sequences as well as any unnecessary flanking sequences. In addition, the recombinant nucleic acid molecule containing the tissue specific promoter and the human class I gene may be isolated from the vector sequences using 1 or more restriction endonucleases. Techniques for manipulating and linearizing recombinant nucleic acid molecules are well known and include the techniques described in Molecular Cloning: A Laboratory Manual, Second Edition, Maniatis et al. eds., Cold Spring Harbor, N.Y. (1989). The linearized recombinant nucleic acid molecule may be microinjected into an egg to produce a genetically altered mammalian egg using well known techniques.

Typically, the linearized nucleic acid molecule is microinjected directly into the pronuclei of the fertilized eggs as has been described by Gordon et al., 1980, PNAS, 77: 7380-7384. This leads to the stable chromosomal integration of the recombinant nucleic acid molecule in a significant population of the surviving embryos. See for example, Brinster et al., 1985, PNAS, 82: 4438-4442 and Hammer et al., 1985, Nature, 315: 600-603. The microneedles used for injection, like the egg holder, can also be pulled from glass tubing. The tip of a microneedle is allowed to fill with plasmid suspension by capillary action. By microscopic visualization, the microneedle is then inserted into the pronucleus of a cell held by the egg holder, and plasmid suspension injected into the pronucleus. If injection is successful, the pronucleus will generally swell noticeably. The microneedle is then withdrawn, and cells which survive the microinjection (e.g. those which do not lyse) are subsequently used for implantation in a host female.

The genetically altered mammalian embryo is then transferred to the oviduct or uterine horns of the recipient. Microinjected embryos are collected in the implantation pipette, the pipette inserted into the surgically exposed oviduct of a recipient female, and the microinjected eggs expelled into the oviduct. After withdrawal of the implantation pipette, any surgical incision can be closed, and the embryos allowed to continue gestation in the foster mother. See, for example, Gordon et al., 1983, Methods in Enzymology, 101: 411; Gordon et al., 1980, PNAS 77: 7390; Hammer et al., 1985, Nature, 315: 600; and Wall et al., 1985, Biol. Reprod., 32: 645.

The host female mammals containing the implanted genetically altered mammalian eggs are maintained for a sufficient time period to give birth to a transgenic mammal having at least 1 cell, e.g. a bone marrow cell, e.g. a hematopoietic cell, which expresses the recombinant nucleic acid molecule of the present invention that has developed from the genetically altered mammalian egg.

At two-four weeks of age (post-natal), tissue samples are taken from the transgenic offspring and digested with Proteinase K. DNA from the samples is phenol-chloroform ID extracted, then digested with various restriction enzymes. The DNA digests are electrophoresed on a Tris-borate gel, blotted on nitrocellulose, and hybridized with a probe consisting of the at least a portion of the coding region of the recombinant cDNA of interest which had been labeled by extension of random hexamers. Under conditions of high stringency, this probe should not hybridize with the endogenous (non-transgene) genes, but should produce a hybridization signal in animals expressing the transgene, allowing for the identification of transgenic pigs.

Production of Transgenic Animals by Cloning

Transgenic animals for use in the present invention may also be made by other methods, for example, by cloning. Cloning by nuclear transfer to enucleated cells is described in U.S. Pat. No. 6,147,276, and in numerous publications, including Campbell et al., 1996, Nature, 380: 64-66; Wilmut et al, 1997, Nature, 385: 810-813; Schneike et al., 1997, Science, 278: 2130-2133; Ashworth et al., 1998, Nature, 394: 329; Sheils et al., 1999, Nature, 399: 316-317; and Evans et al., 1999, Nature Genetics, 23: 90-93.

For example, in order to clone an animal, the following technique may be used. Unfertilised eggs are flushed out of a female animal, which may be induced to produce a larger than normal number of eggs. A sample of tissue is taken from a suitable part of a donor animal (for example, adult tissue such as udder tissue or embryonic tissue) and cultured in vitro. Cultured cells are then starved to send them into a resting or quiescent state by, for example, serum starvation). The donor cell is then fused or injected into the recipient cell. For example, a cell from the culture is placed beside the egg and an electric current used to fuse the couplet. The reconstructed embryo is put into culture and allowed to grow for a length of time (for example, seven days). The recipient cell is activated before, during or after nuclear transfer. Embryos which grow successfully are taken and transferred to a recipient animal which is at the same stage of the oestrus cycle as the egg. The recipient animal becomes pregnant and produces a cloned animal after a suitable gestation period.

Direct microinjection of donor cell nuclei may also be used (the so-called “Honolulu technique”). Direct microinjection of a nucleus from an adult cell into an oocyte from which the nucleus has already been removed has been used to clone mice. The eggs are then prevented from dividing and forming multicelled blastocysts for periods of time (for example, from one to six hours) and subsequently allowed to divide.

Cloning using nuclear transfer from established cell lines is described in Nature 380, 64-66, and also in International Patent Application Numbers PCT/GB96/02099, and PCT/GB96/02098. Transgenic lambs producing recombinant blood clotting factor IX have also been produced. Delayed activation of donor cells is described in UK Patent Numbers GB 2318792 and GB 2340493.

Knock-out Technology

In addition to the addition of exogenous genes to RBCs, a further embodiment of the present invention includes the potential for deletion of genes from RBCs, wherein the deletion provides a therapeutic advantage. For example, it may be advantageous to delete one or more cell surface blood group antigens or epitopes using gene knock out techniques in order to avoid or lessen a host immune response to administered RBCs.

i. Standard Knock Out Animals

Knock out animals are produced by the method of creating gene deletions with homologous recombination. This technique is based on the development of embryonic stem (ES) cells that are derived from embryos, are maintained in culture and have the capacity to participate in the development of every tissue in the animals when introduced into a host blastocyst. A knock out animal is produced by directing homologous recombination to a specific target gene in the ES cells, thereby producing a null allele of the gene. The generation of animals which carry disrupted alleles of GCK/IRS1, IRS1/INSR, MC4R have been described in Huszar et al., 1997, Cell, 88:131. The generation of animals which carry disrupted alleles of BRS3 has been described in Ohki-Hamazaki et al., 1997, Nature, 390: 165. The methodology described in these references can be applied in principal to the creation of any kind of knock out animal.

ii. Tissue Specific Knock Out

The method of targeted homologous recombination has been improved by the development of a system for site-specific recombination based on the bacteriophage P1 site specific recombinase Cre. The Cre-loxP site-specific DNA recombinase from bacteriophage P1 is used in transgenic mouse assays in order to create gene knockouts restricted to defined tissues or developmental stages. Regionally restricted genetic deletion, as opposed to global gene knockout, has the advantage that a phenotype can be attributed to a particular cell/tissue (Marth, 1996, Clin. Invest., 97: 1999). In the Cre-lox-P system one transgenic mouse strain is engineered such that loxP sites flank one or more exons of the gene of interest. Homozygotes for this so called ‘foxed gene’ are crossed with a second transgenic mouse that expresses the Cre gene under control of a cell/tissue type transcriptional promoter. Cre protein then excises DNA between loxP recognition sequences and effectively removes target gene function (Sauer, 1998, Methods, 14: 381). There are now many in vivo examples of this method, including the inducible inactivation of mammary tissue specific genes (Wagner et al., 1997, Nucleic Acids Res., 25: 4323).

iii. Bac Rescue of Knock Out Phenotype

In order to verify that a particular genetic polymorphism/mutation is responsible for altered protein function in vivo one can “rescue” the altered protein function by introducing a wild-type copy of the gene in question. In vivo complementation with bacterial artificial chromosome (BAC) clones expressed in transgenic mice can be used for these purposes. This method has been used for the identification of the mouse circadian Clock gene (Antoch et al., 1997, Cell, 89: 655).

Immunization

The present invention also includes the use of red blood cells comprising an agent in a method of immunization of an animal.

According to this embodiment, an agent comprising an antigen is loaded into a red blood cell as described above. The loaded red blood cell may then be sensitised to render it more susceptible to disruption by exposure to a stimulus than an unsensitised red blood cell. A preferred method of sensitisation is electrosensitisation, as described above. The loaded red blood cell is then introduced into an animal, optionally together with an adjuvant. The animal, or a portion of the animal, is then exposed to an appropriate source of energy to disrupt the red blood cells. Preferably, the source of energy is ultrasound energy, as described in detail above. A single administration/release may be employed, or repeated administrations followed by repeated releases may be employed. The advantage of the immunization regime according to this aspect of the invention is that no immune response is induced in the animal until the agent (antigen) is released. The embodiment comprising repeated release is especially suitable for priming and boosting regimes to ensure a high immune response.

It will be appreciated that the antigens involved need not comprise membrane translocation sequences; indeed, any agent capable of being loaded into a red blood cell is suitable for use as an antigen according to this aspect of the invention.

Kits

A kit designed for the easy delivery of an agent to a recipient vertebrate, whether in a research of clinical setting, is encompassed by the present invention. A kit takes one of several forms, as follows:

A kit for the delivery of an agent to a subject vertebrate comprises preferably sensitised cells, such as red blood cells, and the agent and optionally instructions for loading the agent-MTS conjugate. Alternatively, the red blood cells are supplied loaded with the agent-MTS conjugate for convenience of use by the purchaser. In the latter case, the cells may be supplied in sensitised form, ready for rapid use or pre-sensitised and loaded but needing a final sensitisation step.

The cells of the kit are typically species-specific to the vertebrate of interest, such as a primate, including a human, canine, rodent, mouse, rat, rabbit, sheep, goat, horse, cow, and pig or other, as desired; in other words, the cells are of like species with the intended recipient. In one embodiment, the cells of the kit are, additionally, specific to the blood type of the intended recipient organism, as needed. Optionally, the kit comprises one or more buffers for cell sensitisation, pre-sensitisation, washing, re-suspension, dilution and/or administration to a vertebrate. Appropriate buffers are selected from the group that includes low ionic strength saline, physiological buffers such as PBS or Ringer's solution, cell culture medium and blood plasma or lymphatic fluid. The kit additionally comprises packaging materials (such as tubes, vials, bottles, or sealed bags or pouches) for each individual component and an outer packaging, such as a box, canister or cooler, which contains all of the components of the kit. The kit may be shipped refrigerated. Optionally, non-cellular components are supplied at room temperature or frozen, as needed to maintain their activity during storage and shipping. They may be in liquid or dry (i.e., powder) form.

A second kit of the invention comprises an agent such as a biological effector molecule, instructions for performing the method of the present invention and, optionally a sensitising device and buffers therefor (e.g., saline or other physiological salt buffer, culture medium, plasma or lymphatic fluid). In addition, the kit contains appropriate packaging materials, as described above. The individual components may be supplied in liquid or dry (i.e., powder) form, and may be at room temperature refrigerated or frozen as needed to maintain their activity during storage and shipping. Cells for use with this kit may be obtained independently (for example, they may be harvested from the intended recipient vertebrate).

A preferred aspect of the invention is a kit comprising a red blood cell which is loaded with an agent, and packaging materials therefor. Preferably, a kit as described above further comprises an apparatus for applying the sensitising procedure.

Preferably a kit of the invention further comprises an immunoglobulin or polyethylene glycol. Preferably the kit further comprises a liquid selected from a buffer, diluent or other excipient. More preferably the liquid is selected from a saline buffer, a physiological buffer and plasma.

Another aspect of the invention is a pharmaceutical composition comprising a red blood cell delivery vehicle of the invention comprising an agent such as a biological effector molecule conjugated to an MTS molecule. The red blood cell is admixed with a pharmaceutically acceptable carrier or diluent, or a physiologically compatible buffer. As used herein, the term “physiologically compatible buffer” or “physiological buffer” is defined as a liquid composition which, when placed in contact with living cells, permits the cells to remain alive over a period of minutes, hours or days. As such, a physiological buffer is substantially isotonic with the cell, such that cell volume does not change more than 20% due to differences in internal and external ionic strength. Non-limiting examples of physiologically compatible buffers or physiological buffers include dilute saline, which may be buffered (e.g., Hanks' buffered saline or phosphate buffered saline), or other physiological salts (e.g., Ringer's solution), dilute glucose, sucrose or other sugar, dilute glycerol with or without salts or sugars, cell culture media as are known in the art, serum and plasma.

Preferably, the cell is a human cell. Most preferably, the cell is a red blood cell.

### EXAMPLES

The following examples demonstrate particular embodiments of the invention and are not intended to be limiting.

### Example 1

### Loading Membrane Translocation Sequence Peptides Into Electrosensitised Erythrocytes

The objective of these experiments is to demonstrate that the peptides, penetratin, HIV-TAT and VP22 may be incorporated into electrosensitised erythrocytes. In this study uptake by erythrocytes from a number of sources including human, pig, rabbit and mouse is examined. The penetratin payload comprises a FITC-Penetratin conjugate, having the following sequence: Fluorescein-RQIKIWFQNRRMKWKKC (custom made by Altabioscience, Southampton, UK). The HIV-TAT fragment has the following sequence: Fluorescein-GRKKRRQRRRPPQC-amide (2181.5 Da). VP22 as used here has the following sequence: NAATATRGRSAASRPTERPPAPARSASRPRRPVEC-amide

Whole blood from rabbit is collected in heparinised containers and cells are washed and sensitised as described briefly here. Blood is harvested by venipuncture and washed twice in PBS (phosphate buffered saline) by centrifugation. Cells are suspended in PBS containing 1 mg/ml fluorescein to yield concentrations of 7×10⁸ cells/ml and 0.8 ml aliquots are dispensed into electroporation cuvettes (0.4 cm electrode gap) and retained on ice for 10 min.

The cell concentration is adjusted to 1.5×10⁹ and fluorescein-labelled penetratin, HIV-TAT fragment and VP22 (Alta Biosciences, Edgbaston, Birmingham) are added at the indicated concentrations (in PBS) and mixtures are incubated for 30 min at 37° C.

The mixtures are then centrifuged at 700 g for 5 minutes and the cells are resuspended with PBS-Mg-Glucose (rabbit and mouse) or mBAX (human and pig), and subsequently washed twice. Uptake of peptide is monitored by analysis on flow cytometry where this uptake of the fluorescent peptide is indicated by a shift to the right on the flow cytometry profiles.

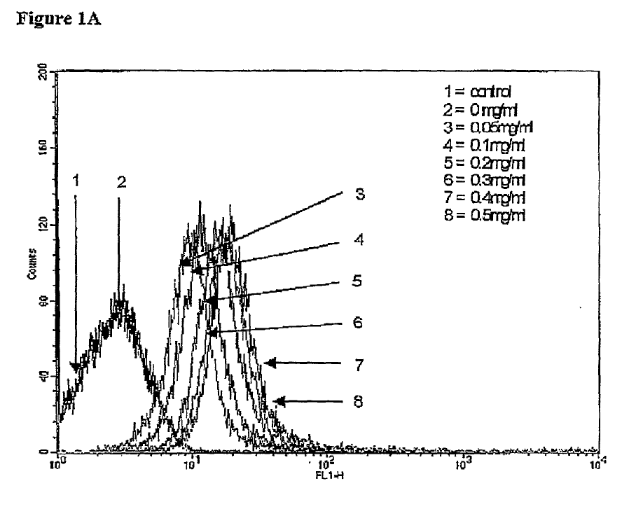

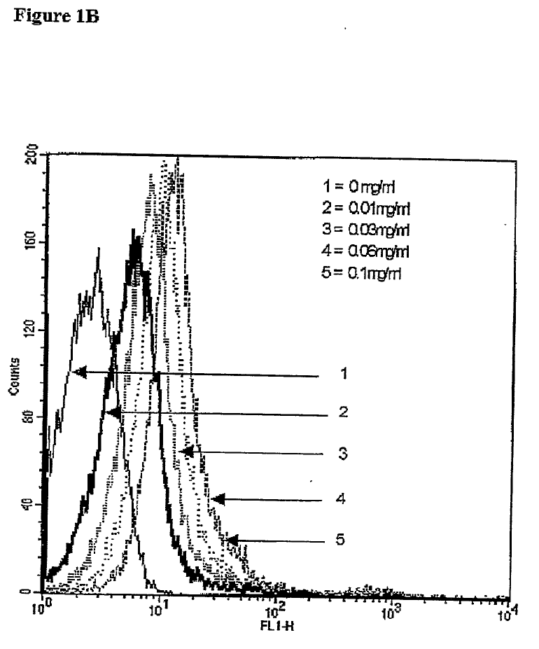

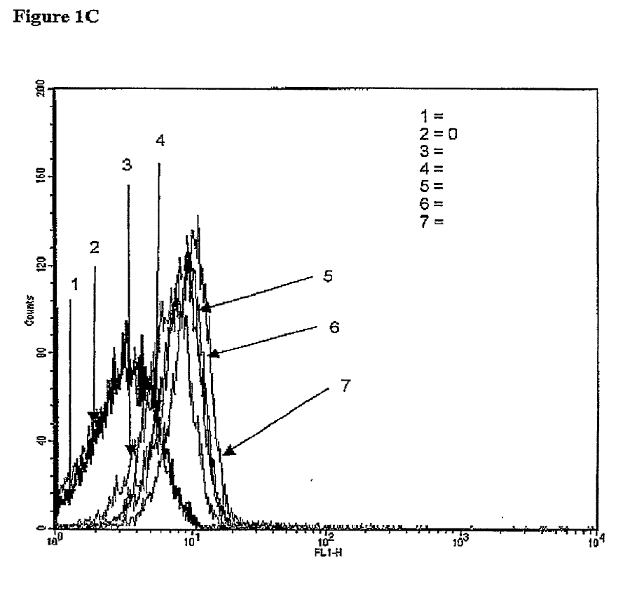

The results are shown in FIG. 1 where increasing concentrations of peptide results in increasing shifts to the right on the flow cytometry profiles (FIG. 1, panels A, B and C). In each case the progressive shift the right is indicative of peptide uptake by the sensitised carrier vehicle. In all cases, the peptide-loaded cells are shown to be preferentially sensitive to low intensity ultrasound (100% lysis when treated with ultrasound at 3 W/cm² and at 1 MHz using the TMM system; TMM is a tissue mimicking material which attenuates ultrasound in the same manner as a soft tissue. The TMM chosen for this work is described in Madsen et al. (1998, Ultrasound Med. & Biol., 24, 535-542) and following preparation, care is taken to ensure that the material has a density of 1.03 g/ml). These results demonstrate that peptides comprising membrane translocation sequences may be auto-loaded into electrosensitised erythrocytes and uptake of peptide is dependant on peptide concentration.

### Example 2

### Stability of Peptide in the Red Blood Cell Vehicle

The objective of these experiments is to demonstrate that, once loaded, the membrane translocation sequence peptides are stable in the vehicle even following incorporation into whole blood.

Erythrocytes loaded with HIV-TAT fragment from human, rabbit, pig and mouse (as above) are spiked (1%) into whole blood of the corresponding species. Stability at 4° C. is assessed for up to 24 hours using flow cytometry, where cells counts of the loaded cell population are analysed against time.

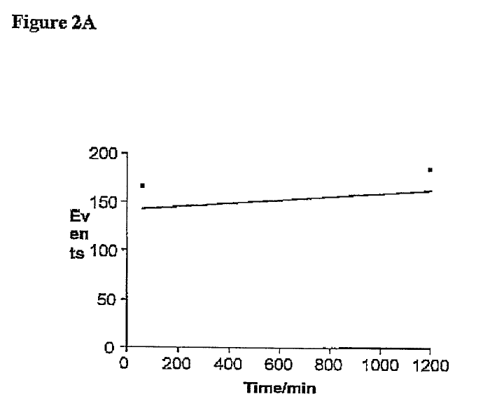

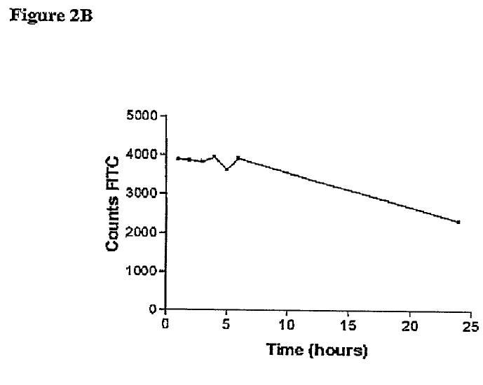

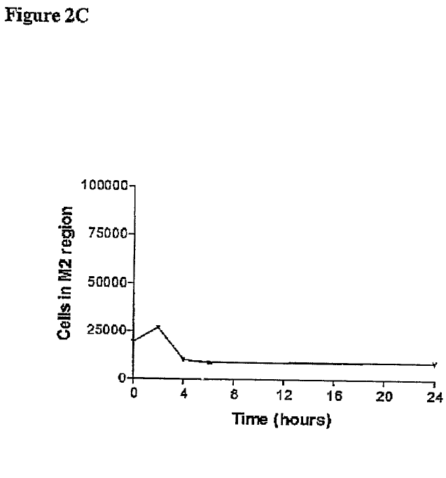

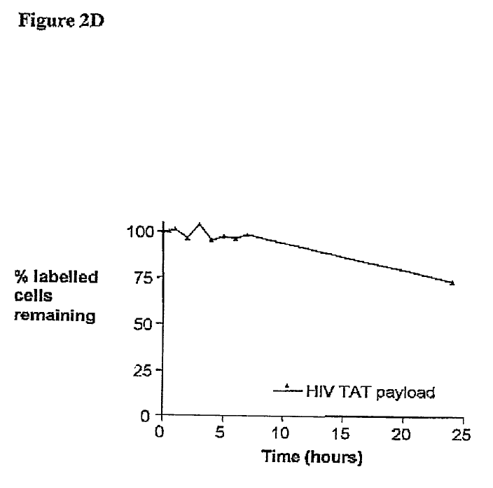

The results are shown in FIG. 2 where loaded human (A), rabbit (B), pig (C) and mouse (D) cells are spiked into whole blood.

In all cases, cell counts of the loaded cell population remain constant throughout the course of the experiment. The results demonstrate that once loaded, the membrane translocation peptides are stable in the vehicle even following incorporation into whole blood.

### Example 3

### Ultrasound-Mediated Release and Bioactivity of the Released Peptide

The objective of these experiments is to demonstrate that the relevant peptide can be released from the vehicle using ultrasound and to further demonstrate that the peptide retained its function in terms of it's ability to enter target cells.

White blood cells are prepared by buoyant density centrifugation Histopaque 1077 (Sigma). Cells are harvested and washed three times in mBax and stored on ice until use. 350 μl of loaded RBC with 0.1 mg/ml penetratin/HIV-TAT fragment are treated on TMM ultrasound 1 MHz Probe, 3 Watts/cm². Samples of the lysates are pooled and centrifuged down to remove debris. WBC populations are incubated together with i) buffer, ii) penetratin and iii) lysates derived from ultrasound treated, penetratin loaded vehicle. Samples are then analysed using flow cytometry and lymphocyte populations are resolved. Uptake of penetratin by this population is indicated by a shift to the right on flow cytometry profiles.

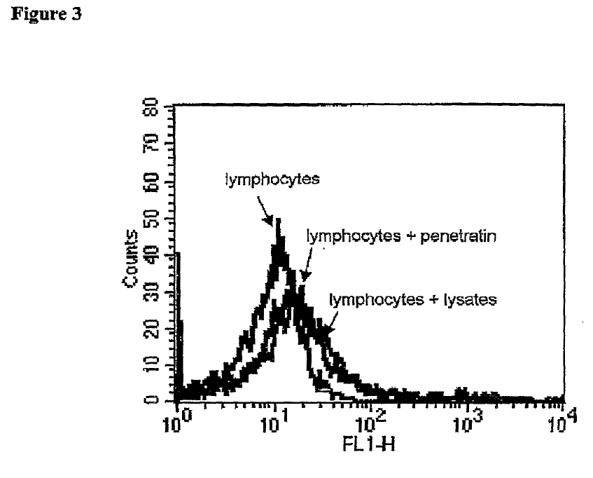

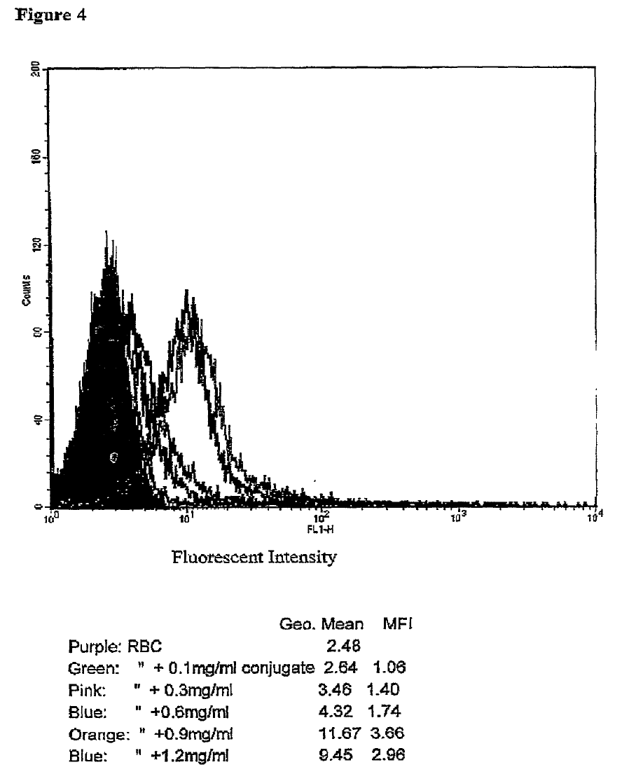

The results are shown in FIG. 3. These illustrate that free penetratin is taken up by the lymphocyte population and the shift of the profile to the right serves as a positive control. In addition, the results demonstrate that penetratin, released from the erythrocyte vehicle, is also taken up by the target lymphcyte population. These results demonstrate that ultrasound facilitates the release of bioactive penetratin from the loaded, sensitised vehicle.

### Example 4

### Ultrasound-Mediated Release and Uptake of the Peptide By Target Endothelium

It has been demonstrated that peptides are capable of trafficking into target cells when released from the vehicle. In terms of exploitation in this invention, the functionality of the peptide will be used to firstly adhere to the endothelium of the vaculature at the target site and secondly to traffic into and beyond the vascular endothelium. In these experiments therefore, a fluorescently-labelled peptide is loaded into sensitised erythrocytes, released from the erythrocytes using ultrasound and the released material be placed in contact with vascular tissue. If the peptide remaines functional following ultrasound-mediated release, then staining of the endothelial cells of the vascular target should be evident.

To the above ends, cells are electrosensitised and loaded with the relevant fluorescently-labelled peptide. The loaded cells are then subjected to ultrasound at 3 W/cm² (in TMM system) and at 1 MHz and the resulting lysates containing the released peptide are retained. A section of aorta is then surgically removed from a rabbit (New Zealand White, female, 2 kg) and perfused thoroughly with phosphate buffered saline (PBS). Sections are ligated at one end. These sections are filled with unloaded erythrocytes; electrosensitised, peptide erythrocytes and lysates resulting from ultrasound treatment of the latter. Each section is retained at room temperature for 30 min. and subsequently flushed with PBS. The tissues are then fixed (40% [v/v] formaldehyde solution in PBS), washed, placed at 4° C. in 30% (w/v) sucrose (in PBS) overnight, sectioned using a cryostat and visualised using fluorescent microscopy.

It is found that in tissues which are placed in contact with either control erythrocytes or electrosensitised, peptide-loaded erythrocytes no fluorescent staining is evident. However, the tissues which are in contact with lysates derived from ultrasound treated, electrosensitised, peptide-loaded erythrocytes, strong fluorescence is evident in endothelial lining and in some cases this fluorescence permeated through to the surrounding tissues. The results demonstrate that the peptide is active in terms of trafficking across cell membranes. The results also suggest that this phenomenon may be exploited to enable uptake of payload conjugates by target tissues following ultrasound-mediated release from the erythrocyte vehicle.

### Example 5

### Uptake of a Penetratin-Phosphorothioate Backbone Oligonucleotide Conjugate by Electrosensitised Erythrocytes

The objective of these experiments is to demonstrate that the an agent-MTS conjugate, namely, an oligonucleotide conjugated to penetratin may be incorporated into electrosensitised erythrocytes. In this study uptake by erythrocytes from human is examined.

Whole blood from human is collected in heparinised containers and cells are washed and sensitised as described above.

The cell concentration is adjusted to 7.0×10⁸ and a FITC-penetratin-phosphorothioate backbone oligonucleode conjugate (Alta Biosciences, Edgbaston, Birmingham) is added at the indicated concentrations (in PBS) and mixtures are incubated for 30 min at 37° C.

The mixtures are then centrifuged at 700 g for 5 minutes and the cells are resuspended with mBAX, and subsequently washed twice. Uptake of peptide is monitored by analysis on flow cytometry where this uptake of the fluorescent peptide is indicated by a shift to the right on the flow cytometry profiles.

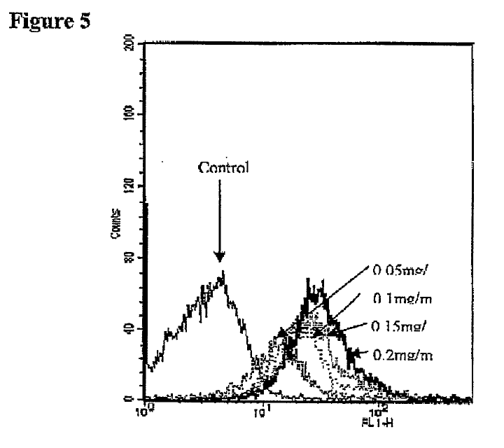

The results are shown in FIG. 5 where increasing concentrations of peptide results in increasing shifts to the right on the flow cytometry profiles. In each case the progressive shift the right is indicative of peptide uptake by the sensitised carrier vehicle. These results demonstrate that membrane translocation sequence peptide conjugates may be auto-loaded into electrosensitised erythrocytes and uptake is dependant on concentration.

Examples 6, 7 and 8 describe loading of FITC-labelled HIV-TAT fragment into electrosensitised erythrocytes by dialysis, ultrasound mediated release of payload in whole circulating blood in vitro, ultrasound mediated release in an in vivo model, and demonstration that circulating cells remain sensitised.

### Example 6

### Loading of FITC-Labelled HI V-TAT Fragment into Electrosensitised Erythrocytes by Hypotonic Dialysis

The objective of these experiments is to demonstrate that the peptide HIV-TAT fragment may be incorporated into electrosensitised erythrocytes using dialysis loading.

Whole blood from pig is collected in heparinised containers and cells are washed and sensitised as described in Example 1 above. The cell concentration is adjusted to 7×10⁸.

Cells are washed once in PBS at 700 g for 5 min, and once in buffer 1 at 700 g for 5 mins. The cells are retained as a packed cell volume and fluorescein-labelled HI V-TAT fragment (Alta Biosciences, Edgbaston, Birmingham) is added to the packed cell volume at the indicated concentrations (expressed as mg/ml peptide to 7×10⁸ cells/ml) and mixtures placed in dialysis tubing (1000 Da MW tubing, Spectro-Por, Spectrum Inc.,) for 60 min at 37° C. Cells are then dialysed against buffer 2 for one hour at 4° C. Membranes are then placed into mBAX at 37° C., and dialysed for one hour.

Cells are harvested from the dialysis membranes and washed three times in mBAX buffer at 170 g for 15 minutes at room temperature. Cells are re-suspended at 7×10⁸ in mBAX and stored at 4° C. o/n.

The next day, cells are washed and sensitised as described above, and the cell concentration is adjusted to 7×10⁸ cells/ml.

Uptake of peptide is monitored by analysis on flow cytometry where this uptake of the fluorescent peptide is indicated by a shift to the right on the flow cytometry profiles.

Erythrocytes loaded with HIV-TAT fragment are spiked (1%) into whole blood. Stability at 37° C. and 4° C. is assessed for up to 24 hours using flow cytometry, where cells counts of the loaded cell population are analysed against time.

The results are shown in FIG. 5 where increasing concentrations of peptide result in increasing shifts to the right on the flow cytometry profiles (FIG. 5). In each case the progressive shift the right is indicative of peptide uptake by the sensitised carrier vehicle. In all cases, the peptide-loaded cells are shown to be preferentially sensitive to low intensity ultrasound (100% lysis when treated with ultrasound at 3 W/cm² and at 1 MHz using a Tissue Mimicking Medium system as described briefly here. In a TMM system, the target is placed at a distance of 1.3 cm from the emitting surface of the ultrasound head and the intervening space is filled with a tissue mimicking material (TMM) which attenuates ultrasound in the same manner as a soft tissue. The TMM chosen for this work is described in Madsen et al. (1998, Ultrasound Med. & Biol., 24, 535-542) and following preparation, care is taken to ensure that the material has a density of 1.03 g/ml.

These results demonstrate that HIV-TAT fragment may be auto-loaded into electrosensitised erythrocytes more effectively by dialysis loading, and uptake of peptide is dependant on peptide concentration.

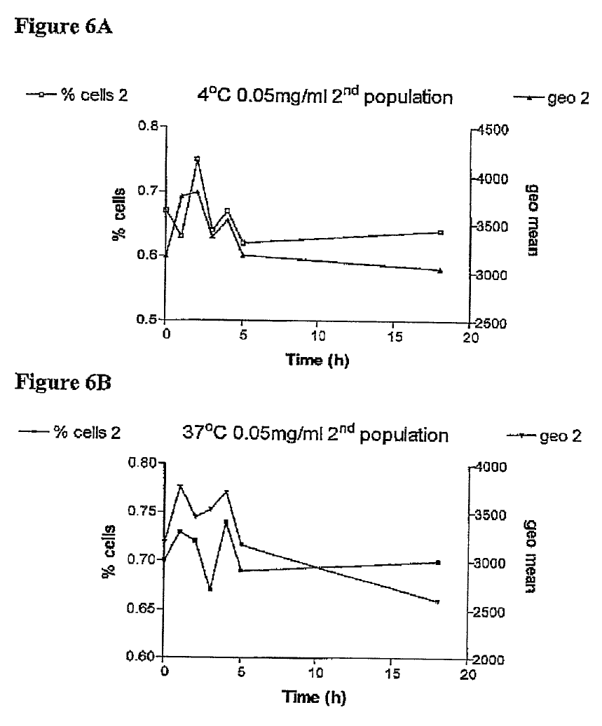

The results are shown in FIG. 6 where loaded pig cells are spiked into whole blood.

In both cases, cell counts of the loaded cell population remain constant throughout the course of the experiment. The results demonstrate that once dialysis loaded, the HIV-TAT fragment is stable in the vehicle even following incorporation into whole blood.

### Example 7

### Ultrasound Mediated Release of Payload in Whole Circulating Blood in Vitro

The objective of this experiment is to demonstrate that the relevant peptide can be released by ultrasound from the vehicle in an in vitro circulating model, at 37° C., 1.3 cm from the ultrasound probe, spiked into whole blood. From this, ultrasound parameters may be established for further use in an in vivo system.

Erythrocytes, electrosensitised and dialysis loaded with HIV-TAT fragment are spiked (2.5%) into whole blood of the same animal. A 3 ml sample is then applied to the circulating phantom model at 4.5-6 W/cm² (pulsed wave; 35%) for 15 min, and 100 μl samples collected for the circulating system every 5 min. Any ultrasound mediated decrease in loaded erythrocytes is demonstrated by loss of cells on the flow cytometer.

Haemoglobin levels in the supernatants of the collected samples are assessed at Abs₅₄₀ₙₘ on the spectrophotometer.

Erythrocytes, non electrosensitised and dialysis loaded with HIV-TAT fragment are spiked (2.5%) into whole blood of the same animal. A 3 ml sample is then applied to the circulating phantom model at 5-8 W/cm² (pulsed wave; 35%) for 15 min. and 100 μl samples collected for the circulating system every 5 min. Haemoglobin levels in the supernatants of the collected samples are then assessed.

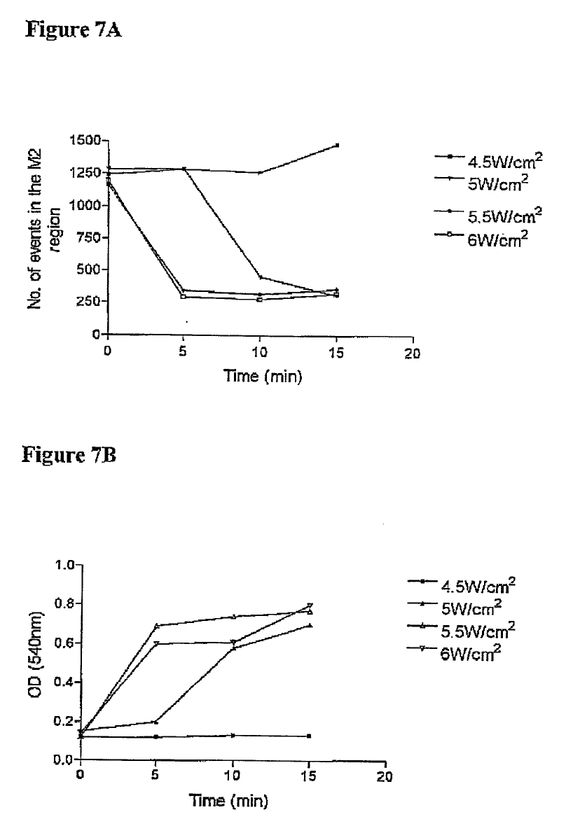

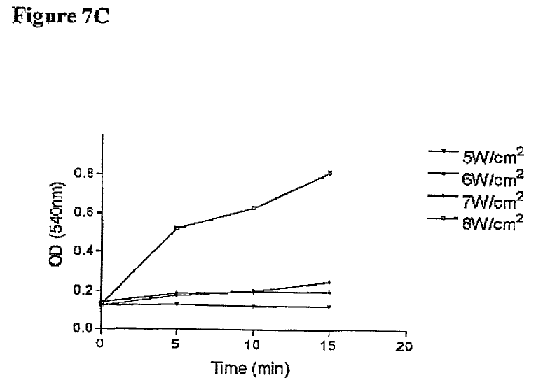

FIG. 7A demonstrates that under the parameters used, an ultrasound intensity of 4.5 W/cm² confers negligible effects on the number of loaded cells in whole blood. At 5 W/cm² a decrease in the number of loaded cells occurs after 10 min, whereas, at 5.5 and 6 W/cm², this time is reduced to 5 minutes.

FIG. 7B demonstrates haemoglobin release at the various ultrasound intensities and shows that release of this cell lysis marker mirrors the loss of labelled cells showing that these cells are being targeted by ultrasound. These results show that in the in vitro circulating system, the loaded vehicle, spiked into whole blood is sensitive to ultrasound.

FIG. 7C illustrates that non electrosensitive, HIV-TAT fragment loaded pig erythrocytes display no changes in haemoglobin release when subjected to conditions of pulsed wave ultrasound at 5-7 W/cm² i.e., no ultrasound mediated lysis of non sensitised cells occurs.

Combined with the information in FIG. 7A, this establishes that a therapeutic window of between 5 to 7 W/cm² may be used in an in vivo model, to induce ultrasound mediated release 1 of peptide payload in electrosensitised loaded cells

### Example 8

### Ultrasound Mediated Release of Payload Release in Vivo, and Demonstration that Circulating Cell Remains Sensitised

The objective of this experiment is to demonstrate that the relevant peptide is released by ultrasound from the vehicle in an in vivo model. Secondly, we investigate whether the loaded cells collected from the circulation still retain ultrasound sensitivity in the in vitro system. The presence of any in vivo repair processes to the loaded vehicle may be identified.

The test system comprised two healthy, mature pigs of a crossbreed type (Large While x Landrace) of the male sex at least four week of age, each weighing 10 kg. Venous puncture of the jugular vein of each animal enables 35 mls of whole blood to be available for processing i.e., electrosensitisation and dialysis loading with fluorescently labelled HIV-TAT fragment. Anaesthesia is induced by injection of pentobarbitone at a dose rate of approx. 25 mg/kg bodyweight (Sagatal (Merial). The exterior ileal vein is catheterised and fitted with a 3 way tap, for sample administration and sampling. Pre-administration samples are collected, prior to the test system receiving the processed packed cells, by slow intravenous injection (5 ml).

In one of the subjects, after 60 min, ultrasound is applied to the jugular/carotid region of the neck at 6 W/cm² (pulsed wave; 35%) (RICH-MAR CRM-1 machine fitted with a 1 mHz head, for 3×10 min bursts. The surface of this area is liberally covered with Alpha Lube gel (Ultrasonic Scanning gel, BCF Technology Ltd) before application of the head.

Samples are collected at the time periods shown, and analysed using flow cytometry, where cells counts of the loaded cell population are assessed.

Additional samples are collected, 10 minutes following cell administration to the animal, for application to the in vitro circulating model. Samples are collected 10 minutes following cell administration, from the circulating system, and assessed using flow cytometry, where cells counts of the loaded cell population are analysed.

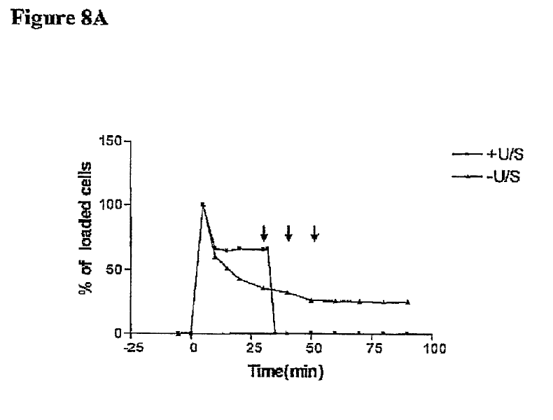

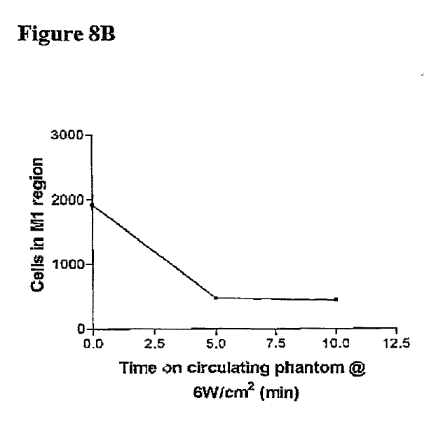

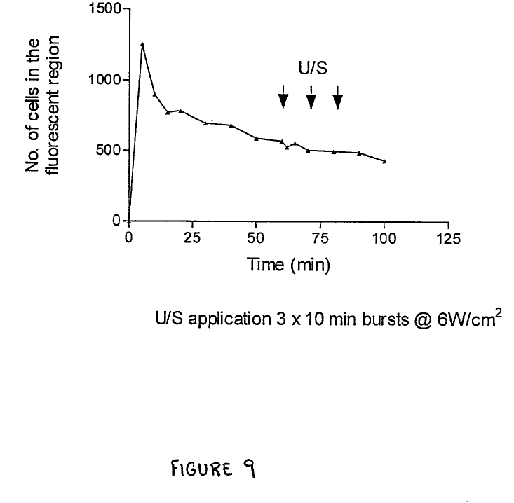

FIG. 8A demonstrates that a clear increase in percentage loaded cells coincides with administration of loaded vehicle into the animal. In both subjects, cell number decreases quite significantly, between 5 and 10 minutes following administration. Spiking of a comparable volume of loaded cells into whole blood however would suggest that the 5-minute sample may not have been an accurate representation, with insufficient dilution of the loaded cells.

In the control animal, to which no ultrasound is applied, a gradual decline in labelled cell number is observed. In contrast, the effect of ultrasound on loaded cells in vivo is pronounced, and a dramatic decrease is shown between 2 and 5 minutes of ultrasound treatment at 6 W/cm², pulsed wave; 35%.

FIG. 8B illustrates that samples collected 10 minutes following cell administration to the animal, for application to the in vitro circulating model, still show a decrease in loaded cell number with ultrasound treatment. This would suggest that in vivo repair processes during circulation are negligible, and the loaded vehicle still demonstrates ultrasound sensitivity.

### Example 9

### Effect on Ultrasound on a Non-Electrosensitised HIV-TAT Loaded Vehicle in Vivo

The objective of this experiment was to demonstrate that loaded vehicle, which had not been electrosensitised would not release its loaded components in vivo.

Whole blood from pig was collected in heparinized containers and cells were washed and sensitised as described in Example 1 of the ultrasound sensitisation filing. The cell concentration was adjusted to 7×10⁸.

Cells were washed once in PBS at 700 g for 5 min, and once in buffer 1 at 700 g for 5 mins. The cells were retained as a packed cell volume and fluorescein-labelled HIV TAT (Alta Biosciences, Edgbaston, Birmingham) was added to the packed cell volume at the indicated concentrations (expressed as mg/ml peptide to 7×10⁸ cells/ml) and mixtures placed in dialysis tubing (1000 Da MW tubing, Spectro-Por, Spectrum Inc.) for 60 min at 37° C. Cells were then dialysed against buffer 2 for one hour at 4° C. Membranes were then placed into mBAX at 37° C., and dialysed for one hour.

Cells were harvested from the dialysis membranes and washed three times in mBAX buffer at 170 g for 15 minutes at room temperature. Cells were resuspended at 7×10⁸ in mBAX and stored at 4° C. overnight.

The next day, cells were washed but not re-sensitised and the cell concentration was adjusted to 7×10⁸ cells/ml.

The in vivo test system comprised of a healthy, mature pig of a crossbreed type (Large While x Landrace) of the male sex at least four week of age, each weighing 10 kg. Venous puncture of the jugular vein of the animal in enabled 35 mls of whole blood to be available for processing i.e. electrosensitisation and dialysis loading with fluorescently labelled HIV-TAT. Anaesthesia was induced by injection of pentobarbitone at a dose rate of approx. 25 mg/kg bodyweight (Sagatal (Merial). The exterior ileal vein was catheterised and fitted with a 3 way tap, for sample administration and sampling. Preadministration samples were collected, prior to the test system receiving the processed packed cells, by slow intravenous injection (5 ml).

In one of the subjects, after 60 min, ultrasound was applied to the jugular/carotid region of the neck at 6 W/cm² (pulsed wave; 35%) (RICH-MAR CRM-1 machine fitted with a 1 mHz head, for 3×10 min bursts. The surface of this area was liberally covered with Alpha Lube gel (Ultrasonic Scanning gel, BCF Technology Ltd) before application of the head. Samples were collected at the time periods shown in FIG. 9, and analyzed using flow cytometry, where cells counts of the loaded cell population were assessed.

FIG. 9 demonstrates that a clear increase in the percent of loaded cells coincides with administration of loaded vehicle into the animal. A gradual decline in labelled cell number was observed, prior to the administration of ultrasound and during and after the 3×10 min bursts, under the parameters used, no effect on cell number could be observed. This would suggest that as the cells were not resensitised, they were not receptive to ultrasound mediated lysis and subsequent release of payload, as would be predicted.

Each of the applications and patents mentioned above, and each document cited or referenced in each of the foregoing applications and patents, including during the prosecution of each of the foregoing applications and patents (“application cited documents”) and any manufacturer's instructions or catalogues for any products cited or mentioned in each of the foregoing applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. A method of preparing a delivery vehicle suitable for delivering an agent to a target site in a vertebrate, the method comprising the steps of:
(a) providing a cell; and
(b) loading the cell with an agent-MTS conjugate, wherein said agent-MTS conjugate comprises a membrane translocation sequence enabling the agent to cross the plasma membrane of a cell, thereby producing the delivery vehicle.

2. A method according to claim 1, which further comprises the step of sensitising the cell, whether before or after the loading step (b), to render the cell more susceptible to disruption by exposure to a stimulus than an unsensitised cell.

3. The method according to claim 1 or 2, wherein the cell is a red blood cell.

4. A method of preparing a delivery vehicle suitable for delivering an agent to a target site in a vertebrate, the method comprising the steps of:
(a) providing a cell loaded with an agent-MTS conjugate, wherein said agent-MTS conjugate comprises a membrane translocation sequence enabling the agent to cross the plasma membrane of a cell; and
(b) sensitising the cell.

5. The method of claim 4, wherein the cell is a red blood cell.

6. A method of preparing a delivery vehicle suitable for delivering an agent to a target site in a vertebrate, the method comprising the steps of:
(a) providing a sensitised cell; and
(b) loading the cell with an agent-MTS conjugate, wherein said agent-MTS conjugate comprises a membrane translocation sequence enabling the agent to cross the plasma membrane of a cell, thereby producing the delivery vehicle.

7. The method according to claim 6, wherein the cell is a red blood cell.

8. A method for delivering an agent to a target site in a vertebrate, comprising the steps of:
(a) providing a cell;
(b) loading the cell with an agent-MTS conjugate, wherein said agent-MTS conjugate comprises a membrane translocation sequence enabling the agent to cross the plasma membrane of a cell;
(c) sensitising the cell to render it more susceptible to disruption than an unsensitised cell;
(d) introducing the cell into a vertebrate; and
(e) causing the agent-MTS conjugate to be released from the sensitised red blood cell by applying energy to the sensitised red blood cell.

9. The method of claim 8, wherein the cell is a red blood cell.

10. The method of claim 8, wherein step (b) is performed prior to step (c).

11. The method of claim 8, wherein step (b) is performed after step (c).

12. A red blood cell vehicle suitable for delivery of an agent to a vertebrate, the red blood cell comprising agent-MTS conjugate, wherein said agent-MTS conjugate comprises a membrane translocation sequence enabling the agent to cross the plasma membrane of a cell, thereby producing the delivery vehicle.

13. The red blood cell vehicle according to claim 12, in which the red blood cell is sensitised so that it is rendered more susceptible to disruption by exposure to a stimulus than an unsensitised red blood cell.

14. The method according to any of claims 2, 4, 6, or 8, where the delivery vehicle is sensitised by applying an electric field to the vehicle.

15. The red blood cell according to claim 13, wherein cell is sensitised by applying an electric field to the red blood cell.

16. The method according to claim 14, wherein the electric field has a field strength of from 0.1 kVolts/cm to 10 kVolts/cm under in vitro conditions.

17. The method according to claim 14, wherein the cell is sensitized by the application of an electric pulse for between 1 μs and 100 milliseconds.

18. The red blood cell according to claim 15, wherein the electric field has a field strength of from 0.1 kVolts/cm to 10 kVolts/cm under in vitro conditions.

19. The red blood cell according to claim 15, wherein the red blood cell is sensitised by application of an electric pulse for between 1 μs and 100 milliseconds.

20. The method according to any of claims 2, 4, 6, or 8, wherein the sensitised red blood cell is disruptable by exposure to ultrasound.

21. The method of claim 20, in which the ultrasound is selected from the group consisting of diagnostic ultrasound, therapeutic ultrasound and a combination of diagnostic and therapeutic ultrasound.

22. The method of claim 21, wherein the applied ultrasound energy source is at a power level of from 0.05 W/cm² to 100 W/cm².

23. The method according to any of claims 2, 4, 6, or 8, in which the cell is pre-sensitised so that it is capable of being loaded with an at least 2-fold greater amount of agent than a cell which has not been pre-sensitised.

24. The method according to claim 23, in which pre-sensitisation comprises exposing the cell to an electric field and/or ultrasound.

25. The method according to any of claims 1, 2, 4, 6, or 8, wherein the agent-MTS conjugate comprises a fusion protein comprising a polypeptide agent fused to a membrane translocation sequence.

26. The method of claim 25, wherein said membrane translocation sequence is selected from the group consisting of: HIV-1-trans-activating protein (Tat), Drosophila Antennapedia homeodomain protein (Antp-HD), Herpes Simplex-1 virus VP22 protein (HSV-VP22), signal-sequence-based peptides, Transportan and Amphiphilic model peptide, homologs, fragments, variants, and mutants thereof having membrane translocational activity.

27. The red blood cell delivery vehicle of claim 12, wherein the agent-MTS conjugate comprises a fusion protein comprising a polypeptide agent fused to a membrane translocation sequence.

28. The red blood cell delivery vehicle of claim 27, wherein said membrane translocation sequence is selected from the group consisting of: HIV-1-trans-activating protein (Tat), Drosophila Antennapedia homeodomain protein (Antp-HD), Herpes Simplex-1 virus VP22 protein (HSV-VP22), signal-sequence-based peptides, Transportan and Amphiphilic model peptide, homologs, fragments, variants, and mutants thereof having membrane translocational activity.

29. The red blood cell delivery vehicle of claim 12, wherein the cell is presensitized, such that the cell comprise at least twice as much of an agent-MTS conjugate as a non-presensitized loaded cell.

30. The method according to any of claims 1, 2, 4, 6, or 8, wherein the agent-MTS conjugate comprises the membrane translocation sequence GRKKRRQRRRPPQC, RQIKIWFQNRRMKWKK or RQIKIWFQNRRMKWKKC.

31. The red blood cell delivery vehicle of claim 12, wherein the agent-MTS conjugate comprises the membrane translocation sequence GRKKRRQRRRPPQC, RQIKIWFQNRRMKWKK or RQIKIWFQNRRMKWKKC.

32. The method of any of claims 1, 2, 4, 6, or 8, wherein the agent is selected from a group consisting of a biologically active molecule, a protein, a polypeptide, a peptide, a nucleic acid, a virus-like particle, a nucleotide, a ribonucleotide, a deoxyribonucleotide, a modified deoxyribonucleotide, a heteroduplex, a nanoparticle, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a carbohydrate, and mixtures, fusions, combinations or conjugates thereof.

33. The red blood cell delivery vehicle of claim 12, wherein the agent is selected from a group consisting of a biologically active molecule, a protein, a polypeptide, a peptide, a nucleic acid, a virus-like particle, a nucleotide, a ribonucleotide, a deoxyribonucleotide, a modified deoxyribonucleotide, a heteroduplex, a nanoparticle, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a carbohydrate, and mixtures, fusions, combinations or conjugates thereof.

34. The method of any of claims 1, 2, 4, 6, or 8, wherein the agent is chemically bonded to, fused to, mixed with, or combined with, an imaging agent.

35. The red blood cell delivery vehicle of claim 12, wherein the agent is chemically bonded to, fused to, mixed with, or combined with, an imaging agent.

36. A kit comprising a red blood cell, an agent-MTS conjugate comprising a membrane translocation sequence suitable for loading into said red blood cell, and packaging materials therefor.

37. The kit according to claim 36, in which the agent-MTS conjugate is loaded into the red blood cell.

38. The kit according to claim 36 or 37, in which the cell is sensitised.

39. The kit according to claim 36 or 37, in which the cell is pre-sensitised.

40. A pharmaceutical composition comprising the red blood cell delivery vehicle of claim 12, and a physiologically compatible buffer.

41. A method of loading a red blood cell with an agent, the method comprising the steps of:
(a) providing a red blood cell; and
(b) exposing the red blood cell to an agent-MTS conjugate, wherein said agent-MTS conjugate comprises a membrane translocation sequence enabling the agent to cross the plasma membrane of a cell, for a suitable period of time until said red blood cell is loaded with said agent.

42. A method of loading a red blood cell with an agent, the method comprising the steps of:
(a) providing a red blood cell;
(b) providing an agent to be delivered;
(c) joining the agent to a membrane translocation sequence enabling the agent to cross the plasma membrane of a cell, thereby forming an agent-MTS conjugate; and
(d) exposing the red blood cell to the agent-MTS conjugate, for a suitable period of time until said red blood cell is loaded with said agent.

43. A method of immunisation of an animal with an antigen, the method comprising the steps of:
(a) providing a red blood cell;
(b) loading the red blood cell with an antigen;
(c) introducing the red blood cell into a vertebrate; and
(d) causing the agent to be released from the red blood cell.

44. The method according to claim 42, in which the red blood cell is sensitised to render the red blood cell more susceptible to disruption by exposure to a stimulus than an unsensitised red blood cell.

45. The method according to claim 43, wherein the cell is electrosensitised.

46. The method according to claim 43, wherein the red blood cell is disrupted by exposure to ultrasound.

47. The method according to claim 43, in which steps (c) and/or (d) are repeated.
